(19) United States
(12) Patent Application Publication
(10) Pub. No.: US 2025/0295832 A1
(43) Pub. Date: Sep. 25, 2025

(54) IMPLANTABLE MEMBRANE CONSTRUCT AND ENCAPSULATION DEVICES INCORPORATING THE SAME

(71) Applicants: W. L. Gore & Associates, Inc., Newark, DE (US); ViaCyte, Inc., San Diego, CA (US)

(72) Inventors: Timothy Bruhn, Newark, DE (US); Niki FRANGAKIS Decker, Newark, DE (US); Karim Dudum, San Diego, CA (US); Evert Kroon, San Diego, CA (US); Jeffery Leid, Newark, DE (US); Craig McGreevy, San Diego, CA (US); Jonathan Valdez, San Diego, CA (US); Lauren Zambotti, Newark, DE (US); Janahan Arumoli, San Diego, CA (US)

(21) Appl. No.: 19/116,532

(22) PCT Filed: Sep. 29, 2023
(86) PCT No.: PCT/US2023/075582

§ 371 Date: Mar. 28, 2025

Related U.S. Application Data
(60) Provisional application No. 63/412,209, filed on Sep. 30, 2022.

(51) Int. Cl. A61L 27/16; A61L 27/56

(52) U.S. Cl. CPC A61L 27/16; A61L 27/56; A61L 2300/606; A61L 2300/62

(57) ABSTRACT

An implantable membrane construct including a maximum pore size (MPS) less than 2 microns and opposing sides, each side of the construct having a surface roughness (Sa) greater than about 0.5 microns is disclosed. When the luminal surface of the implantable membrane construct has a surface roughness (Sa) greater than about 0.5 microns, mesenchymal cells do not form at the interface of the lumen and the first layer (i.e., luminal interface) such that the mesenchymal cells do not impede the flow of oxygen and nutrients to the graft cells (when implanted). When implanted, the outermost layer of the implantable membrane construct enables cellular penetration, vascularization, and anchoring of the construct. The implantable membrane construct includes single layer embodiments and multiple layer embodiments. Encapsulation devices utilizing the implantable membrane constructs to encapsulate biological entities (e.g., cells) into a patient are also provided.

### FIELD

The present disclosure relates generally to the field of implantable medical devices and, in particular, to an implantable membrane construct and encapsulation devices containing the same.

### BACKGROUND

Biological therapies, including cellular derived therapies, are increasingly viable methods for treating chronic and debilitating diseases in humans.

With respect to biological therapies in general, cells, viruses, viral vectors, bacteria, proteins, antibodies, and other bioactive entities may be introduced into a patient by surgical or interventional methods that place the bioactive entities into a patient. Often the bioactive entities are first placed in a device that is then inserted into the patient. Alternatively, the device may be first inserted into a patient with the bioactive entity added later. The device may be formed of one or more implantable membranes or implantable membrane constructs that permit the passage of nutrients through the device but prevent the passage of cells from the device into the patient.

To maintain a viable and productive population of bioactive entities (e.g., cells), the bioactive entities must maintain access to nutrients, such as oxygen, which are primarily delivered through the blood vessels of the host. To maximize the viability and productivity of the implanted, encapsulated cells, it is necessary to maximize access to oxygen and nutrients by ensuring that the formation of blood vessels be as close as possible to the cells such that the diffusion distance and time needed for transport of the oxygen and nutrients to the implanted, encapsulated cells is minimized.

The implantation of external devices, such as, for example, cell encapsulation devices, into a body triggers a foreign body reaction to said device which, depending upon material selection and device design, can lead to deleterious biological reactions. Even when materials/devices are purposefully designed such that the foreign body reaction is minimal, their payload(s), e.g., cells (or other biological moieties) may be adversely affected by myriad factors. In the case of cell therapies (e.g.), one possible outcome could be the selection/preference for non-functional/non-therapeutic cell populations versus a targeted stem cell therapy where the majority of the therapeutic cell population is present and functional. For example, the formation/selection of non-targeted cell types, for example mesenchymal cells, within a population of stem cells may reduce the overall number of therapeutically functional cells (non-mesenchyme) while also increasing the diffusional barrier/distance between the host and the functional/therapeutic cell population. These non-therapeutically functional cells may also physically take up space within the graft/device, reducing the overall efficacy of the therapy.

There is a need in the art for a material that can reduce or even prevent the formation of mesenchymal cells at the luminal interface, and throughout the graft, to permit the encapsulated, therapeutic cells to survive and secrete therapeutically useful substance(s).

### SUMMARY

In one Aspect (“Aspect 1”), an implantable membrane construct includes a first layer having a maximum pore size (MPS) less than about 2 microns and opposing sides, where each opposing side has a surface roughness of at least 0.5 microns.

According to another Aspect (“Aspect 2”) further to Aspect 1, where the first layer includes a polymer membrane.

According to another Aspect (“Aspect 3”) further to Aspect 2, where the polymer membrane is a fluoropolymer membrane.

According to another Aspect (“Aspect 4”) further to Aspect 3, where the fluoropolymer membrane includes expanded polytetrafluoroethylene (ePTFE), a modified ePTFE membrane, a tetrafluoroethylene (TFE) copolymer membrane, a polyvinylidene fluoride (PVDF) membrane, or a fluorinated ethylene propylene (FEP) membrane.

According to another Aspect (“Aspect 5”) further to Aspect 1, including a surface coating at least partially on the first layer, wherein the surface coating includes one or more members selected from antimicrobial agents, antibodies, pharmaceuticals, biologically active molecules, and a hydrophilic coating.

According to another Aspect (“Aspect 6”) further to Aspect 1, including a frame positioned around the perimeter of the first layer.

According to another Aspect (“Aspect 7”) further to Aspect 1, including multiple regions, where each region has a microstructure.

In one Aspect (“Aspect 8”), an implantable membrane construct includes a first layer, a second layer having a maximum pore size less than about 2.0 microns, the second layer being positioned on the first layer, where the first layer has a first externally facing side and the second layer has a second externally facing side, and where the first externally facing side and the second externally facing side each has a surface roughness (Sa) of at least 0.5 microns.

According to another Aspect (“Aspect 9”) further to Aspect 8, where the first layer is cell permeable, and the second layer is cell impermeable.

According to another Aspect (“Aspect 10”) further to Aspect 8, where the membrane construct has a total thickness from about 15 microns to about 150 microns.

According to another Aspect (“Aspect 11”) further to Aspect 8, including a mean flow pore size greater than about 0.1 microns.

According to another Aspect (“Aspect 12”) further to Aspect 8, where the tensile strength in the weakest direction is greater than about 0.15 N/mm.

According to another Aspect (“Aspect 13”) further to Aspect 8, including a reinforcing component.

According to another Aspect (“Aspect 14”) further to Aspect 13, where the reinforcing component has a stiffness from about 0.01 N/cm to about 5 N/cm.

According to another Aspect (“Aspect 15”) further to Aspect 13, where the reinforcing component is a woven or non-woven textile.

According to another Aspect (“Aspect 16”) further to Aspect 8, where at least one of the first layer and the second layer includes a polymer, a fluoropolymer membrane, a non-fluoropolymer membrane, a woven textile, a non-woven textile, woven or non-woven collections of fibers or yarns, fibrous matrices, and combinations thereof.

According to another Aspect (“Aspect 17”) further to Aspect 16, where the polymer is a fluoropolymer membrane selected from an expanded polytetrafluoroethylene (ePTFE) membrane, a fluorinated ethylene propylene (FEP) membrane, and a modified ePTFE membrane.

According to another Aspect (“Aspect 18”) further to Aspect 8, where the first layer and the second layer are intimately bonded.

According to another Aspect (“Aspect 19”) further to Aspect 8, where the coefficient of variation of the surface roughness (Sa) of opposing sides in the implantable membrane construct is less than 15%.

In one Aspect (“Aspect 20”), an implantable membrane construct includes a first layer having a first externally facing side with a surface roughness (Sa) of at least 0.5 microns, a second layer positioned on the first layer on a side opposing the first externally facing side, and a third layer positioned on the second layer such that the second layer is positioned between the first layer and the third layer, where the third layer has a second externally facing side having a surface roughness (Sa) of at least 0.5 microns, and where the implantable membrane has a maximum pore size less than about 2.0 microns.

According to another Aspect (“Aspect 21”) further to Aspect 20, where the first layer is cell permeable and the second and third layers are cell impermeable.

According to another Aspect (“Aspect 22”) further to Aspect 20, including a mean flow pore size greater than about 0.1 microns.

According to another Aspect (“Aspect 23”) further to Aspect 20, where the first layer has a thickness from about 2 microns to about 100 microns.

According to another Aspect (“Aspect 24”) further to Aspect 20, where the surface roughness (Sa) of the first layer and the third layer has a coefficient of variation that is less than 15%.

According to another Aspect (“Aspect 25”) further to Aspect 20, where the feature spacing in the third layer is greater than about 3 microns.

According to another Aspect (“Aspect 26”) further to Aspect 20, where the third layer comprises at least one of woven fabrics, non-woven fabrics, and non-fluoropolymer membranes.

According to another Aspect (“Aspect 27”) further to Aspect 20, containing perforations therein.

According to another Aspect (“Aspect 28”) further to Aspect 20, including a tensile strength in the weakest direction greater than about 0.15 N/mm.

According to another Aspect (“Aspect 29”) further to Aspect 20, where at least two of the first layer, the second layer, and the third layer are intimately bonded.

According to another Aspect (“Aspect 30”) further to Aspect 20, including a reinforcing component.

According to another Aspect (“Aspect 31”) further to Aspect 30, where the reinforcing component has a stiffness from about 0.01 N/cm to about 5 N/cm.

According to another Aspect (“Aspect 32”) further to Aspect 30, where the reinforcing component comprises a woven or non-woven textile.

According to another Aspect (“Aspect 33”) further to Aspect 20, where at least one of the first layer, the second layer, and the third layer includes a polymer, a fluoropolymer membrane, a non-fluoropolymer membrane, a woven textile, a non-woven textile, woven or non-woven collections of fibers or yarns, fibrous matrices, and combinations thereof.

According to another Aspect (“Aspect 34”) further to Aspect 33, where the polymer is a fluoropolymer membrane selected from an expanded polytetrafluoroethylene (ePTFE) membrane, a fluorinated ethylene propylene (FEP) membrane, and a modified ePTFE membrane.

According to another Aspect (“Aspect 35”) further to Aspect 20, where the implantable membrane construct has at least partially thereon a surface coating including one or more members selected from antimicrobial agents, antibodies, pharmaceuticals, biologically active molecules, and a hydrophilic coating.

According to another Aspect (“Aspect 36”) further to Aspect 20, where at least one of the first layer, second layer, third layer or reinforcing component is formed of a non-woven fabric.

In one Aspect (“Aspect 37”), a cell encapsulation device includes an implantable membrane construct having a luminal interface with a surface roughness of at least 0.5 microns, an exterior surface with the surface roughness of at least 0.5 microns, and a maximum pore size (MPS) of less than about 2 microns.

According to another Aspect (“Aspect 38”) further to Aspect 37, where the implantable membrane construct includes a first layer.

According to another Aspect (“Aspect 39”) further to Aspect 38, where the first layer includes a polymer membrane.

According to another Aspect (“Aspect 40”) further to Aspect 39, where the polymer membrane is a fluoropolymer membrane.

According to another Aspect (“Aspect 41”) further to Aspect 40, where the fluoropolymer membrane includes an expanded polytetrafluoroethylene (ePTFE) membrane, a modified ePTFE membrane, a tetrafluoroethylene (TFE) copolymer membrane, a polyvinylidene fluoride (PVDF) membrane, or a fluorinated ethylene propylene (FEP) membrane.

According to another Aspect (“Aspect 42”) further to Aspect 37, including a surface coating at least partially on the implantable membrane construct, where the surface coating includes one or more members selected from antimicrobial agents, antibodies, pharmaceuticals, biologically active molecules, and a hydrophilic coating.

According to another Aspect (“Aspect 43”) further to Aspect 37, including a hydrophilic coating on the implantable membrane construct.

According to another Aspect (“Aspect 44”) further to Aspect 37, including a reinforcing component.

According to another Aspect (“Aspect 45”) further to Aspect 43, where the reinforcing component comprises a woven or non-woven textile.

According to another Aspect (“Aspect 46”) further to Aspect 37, including a first layer and a second layer positioned on the first layer, where a luminal interface of the first layer has the surface roughness of at least 0.5 microns and an externally facing side of the second layer has the surface roughness of at least 0.5 microns.

According to another Aspect (“Aspect 47”) further to Aspect 46, where the first layer is cell permeable, and the second layer is cell impermeable.

According to another Aspect (“Aspect 48”) further to Aspect 46, where the membrane construct has a total thickness from about 15 microns to about 150 microns.

According to another Aspect (“Aspect 49”) further to Aspect 46, including a mean flow pore size greater than about 0.1 microns.

According to another Aspect (“Aspect 50”) further to Aspect 46, where the tensile strength in the weakest direction is greater than about 0.15 N/mm.

According to another Aspect (“Aspect 51”) further to Aspect 46, including a reinforcing component.

According to another Aspect (“Aspect 52”) further to Aspect 51, where the reinforcing component is a woven or non-woven textile.

According to another Aspect (“Aspect 53”) further to Aspect 46, where at least one of the first layer and the second layer includes a polymer, a fluoropolymer membrane, a non-fluoropolymer membrane, a woven textile, a non-woven textile, woven or non-woven collections of fibers or yarns, fibrous matrices, and combinations thereof.

According to another Aspect (“Aspect 54”) further to Aspect 46, where the polymer is a fluoropolymer membrane selected from an expanded polytetrafluoroethylene (ePTFE) membrane, a fluorinated ethylene propylene (FEP) membrane, and a modified ePTFE membrane.

According to another Aspect (“Aspect 55”) further to Aspect 46, where the first layer and the second layer are intimately bonded.

According to another Aspect (“Aspect 56”) further to Aspect 46, including a first layer, a second layer, and a third layer, where the second layer is positioned between the first layer and the third layer, and where the luminal interface of the first layer has the surface roughness of at least 0.5 microns and an externally facing side of the third layer has the surface roughness of at least 0.5 microns.

According to another Aspect (“Aspect 57”) further to Aspect 56, where the first layer is cell impermeable and the second and third layers are cell permeable.

According to another Aspect (“Aspect 58”) further to Aspect 56, including a mean flow pore size greater than about 0.1 microns.

According to another Aspect (“Aspect 59”) further to Aspect 56, where the first layer has a thickness from about 2 microns to about 100 microns.

According to another Aspect (“Aspect 60”) further to Aspect 56, where the coefficient of variation of the surface roughness (Sa) of opposing sides of an implantable membrane construct is less than 15%.

According to another Aspect (“Aspect 61”) further to Aspect 56, where the average feature spacing in the third layer is greater than about 3 microns.

According to another Aspect (“Aspect 62”) further to Aspect 56, where the third layer includes at least one of woven fabrics, non-woven fabrics, and non-fluoropolymer membranes.

According to another Aspect (“Aspect 63”) further to Aspect 56, containing perforations therein.

According to another Aspect (“Aspect 64”) further to Aspect 56, including a tensile strength in the weakest direction greater than about 0.15 N/mm.

According to another Aspect (“Aspect 65”) further to Aspect 56, where at least two of the first layer, the second layer, and the third layer are intimately bonded.

According to another Aspect (“Aspect 66”) further to Aspect 56, including a reinforcing component.

According to another Aspect (“Aspect 67”) further to Aspect 66, where the reinforcing component has a stiffness from about 0.01 N/cm to about 5 N/cm.

According to another Aspect (“Aspect 68”) further to Aspect 66, where the reinforcing component comprises a woven or non-woven textile.

According to another Aspect (“Aspect 69”) further to Aspect 56, where at least one of the first layer, the second layer, and the third layer includes a polymer, a fluoropolymer membrane, a non-fluoropolymer membrane, a woven textile, a non-woven textile, woven or non-woven collections of fibers or yarns, fibrous matrices, and combinations thereof.

According to another Aspect (“Aspect 70”) further to Aspect 69, where the polymer is a fluoropolymer membrane selected from an expanded polytetrafluoroethylene (ePTFE) membrane, a fluorinated ethylene propylene (FEP) membrane, and a modified ePTFE membrane.

According to another Aspect (“Aspect 71”) further to Aspect 56, where the implantable membrane construct has at least partially thereon a surface coating including one or more members selected from antimicrobial agents, antibodies, pharmaceuticals, biologically active molecules, and a hydrophilic coating.

According to another Aspect (“Aspect 72”) further to Aspect 56, where at least one of the first layer, second layer, third layer or reinforcing component is formed of a non-woven fabric.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a schematic illustration of a side view of a single layer implantable membrane construct in accordance with some embodiments;

FIG. 2 is a schematic illustration of a side view of a two-layer implantable membrane construct including a mesenchymal mitigation layer and a cell impermeable layer in accordance with some embodiments;

FIG. 2A is a schematic illustration of a side view of a two-layer implantable membrane construct including a cell impermeable layer and a vascularization layer in accordance with some embodiments;

FIG. 3 is a schematic illustration of a side view of a multilayer implantable membrane composite in accordance with some embodiments;

FIG. 4A is a schematic illustration of a top view of a cell encapsulation device in accordance with some embodiments;

FIG. 4B is a schematic illustration of a cross-section of the cell encapsulation device of FIG. 4A depicting the orientation of the layers of the biocompatible membrane composite and placement of cells in accordance with some embodiments;

FIG. 5 is a schematic illustration depicting the determination of feature spacing where three neighboring features represent the corners of a triangle whose circumcircle has an interior devoid of additional features and the feature spacing is the straight distance between two of the features forming the triangle in accordance with some embodiments;

FIG. 6 is a scanning electron micrograph (SEM) of the surface of the ePTFE membrane of Comparative Example 1 in accordance with some embodiments;

FIG. 6A is a scanning electron micrograph (SEM) of the cross-section of the ePTFE membrane of Comparative Example 1 in accordance with some embodiments;

FIG. 7 is a representative histological image depicting mesenchymal cells lining the luminal interface of the implantable membrane construct of Comparative Example 1 in accordance with some embodiments;

FIG. 8 is a scanning electron micrograph (SEM) of the lumen facing side of the implantable membrane construct of Comparative Example 2 in accordance with some embodiments;

FIG. 9 is a scanning electron micrograph (SEM) of the host facing side of the implantable membrane construct of Comparative Example 2 in accordance with some embodiments;

FIG. 9A is a scanning electron micrograph (SEM) of the cross-section of the implantable membrane construct of Comparative Example 2 in accordance with some embodiments;

FIG. 10 is a representative histological image depicting the presence of mesenchymal cells lining the luminal interface of the implantable membrane construct of Comparative Example 2 in accordance with some embodiments;

FIG. 11 is a scanning electron micrograph (SEM) of the lumen facing side of the implantable membrane construct of Example 1 in accordance with some embodiments;

FIG. 12 is a scanning electron micrograph (SEM) of the host facing side of the implantable membrane construct of Example 1 in accordance with some embodiments;

FIG. 13 is a scanning electron micrograph (SEM) of a cross-section of the implantable membrane composite of Example 1 in accordance with some embodiments;

FIG. 14 is a representative histological image depicting the presence of viable functional cells adjacent to the luminal interface of the implantable membrane construct of Example 1 in accordance with some embodiments;

FIG. 15 is a scanning electron micrograph (SEM) of the lumen facing side of the implantable membrane composite of the implantable membrane composite of Example 2 in accordance with some embodiments;

FIG. 16 is a scanning electron micrograph (SEM) of the host facing side of the implantable membrane composite of the implantable membrane composite of Example 2 in accordance with some embodiments;

FIG. 17 is a scanning electron micrograph (SEM) of a cross-section of the implantable membrane construct of Example 2 in accordance with some embodiments;

FIG. 18 is a representative histological image depicting the presence of viable functional cells adjacent to the luminal interface of the implantable membrane composite of Example 2 in accordance with some embodiments;

FIG. 19 is a scanning electron micrograph (SEM) of the lumen facing side of the implantable membrane composite of the implantable membrane composite of Example 3 in accordance with some embodiments;

FIG. 20 is a scanning electron micrograph (SEM) of the host facing side of the implantable membrane composite of the implantable membrane composite of Example 3 in accordance with some embodiments;

FIG. 21 is a scanning electron micrograph (SEM) of a cross-section of the implantable membrane construct of Example 3 in accordance with some embodiments;

FIG. 22 is a representative histological image depicting the presence of viable functional cells adjacent to the luminal interface of the implantable membrane composite of Example 3 in accordance with some embodiments;

FIG. 23 is a scanning electron micrograph (SEM) of the lumen facing side of the implantable membrane composite of the implantable membrane composite of Example 4 in accordance with some embodiments;

FIG. 24 is a scanning electron micrograph (SEM) of the host facing side of the implantable membrane composite of the implantable membrane composite of Example 4 in accordance with some embodiments;

FIG. 25 is a scanning electron micrograph (SEM) of a cross-section of the implantable membrane construct of Example 4 in accordance with some embodiments; and

FIG. 26 is a representative histological image depicting the presence of viable functional cells adjacent to the luminal interface of the implantable membrane composite of Example 4 in accordance with some embodiments.

Persons skilled in the art will readily appreciate that various aspects of the present disclosure can be realized by any number of methods and apparatus configured to perform the intended functions. It should also be noted that the accompanying drawing figures referred to herein are not necessarily drawn to scale but may be exaggerated to illustrate various aspects of the present disclosure, and in that regard, the drawing figures should not be construed as limiting.

### DETAILED DESCRIPTION

Persons skilled in the art will readily appreciate that various aspects of the present disclosure can be realized by any number of methods and apparatus configured to perform the intended functions. It should also be noted that the accompanying figures referred to herein are not necessarily drawn to scale and may be exaggerated to illustrate various aspects of the present disclosure, and in that regard, the figures should not be construed as limiting. Directional references such as “up,” “down,” “top,” “left,” “right,” “front,” and “back,” among others are intended to refer to the orientation as illustrated and described in the figure (or figures) to which the components and directions are referencing. It is to be noted that all ranges described herein are exemplary in nature and include any and all values in between. In addition, all references cited herein are incorporated by reference in their entireties. In addition, the terms “implantable membrane construct”, “membrane construct”, and “construct” may be used interchangeably herein and may refer to one or a plurality of layers. The terms “cell encapsulation device”, “encapsulation device”, and “device” may be interchangeably used herein.

The present disclosure is directed to an implantable membrane construct and encapsulation devices that incorporate the implantable membrane construct. The implantable membrane construct includes a maximum pore size (MPS) that is less than 2.0 microns and opposing sides, where each opposing side has a surface roughness (Sa) that is greater than about 0.5 microns. As used herein, the term “implantable membrane construct” includes a single layer embodiment and multilayer embodiments. In single layer and multilayer embodiments, each side of the implantable membrane construct has a surface roughness (Sa) greater than about 0.5 microns and the implantable membrane construct has a maximum pore size (MPS) less than about 2.0 microns. In such embodiments, a first side of the membrane construct may function as a mesenchymal mitigation layer and an opposing, second side enables cellular penetration, vascularization, and anchoring of the construct (when implanted). It is to be appreciated that a membrane construct with a single layer can have multiple regions throughout the construct with different microstructures.

The implantable membrane construct may also include multiple layers (i.e., more than one layer). In at least one embodiment, a second layer is positioned on a first layer. The externally facing side of the first layer has a surface roughness (Sa) greater than about 0.5 microns and the opposing side of the membrane construct (e.g., the externally facing side of the second layer) also has a surface roughness (Sa) greater than about 0.5 microns. As used herein, the term “externally facing side” is meant to denote the outermost portion of the layer(s) in the implantable membrane construct. When implanted, the externally facing side of the first layer may face the lumen and the externally facing side of the second layer may face the host tissue. Either the first layer or the second layer provides cell impermeability to the membrane construct. The implantable membrane construct has a maximum pore size (MPS) less than about 2.0 microns.

In certain embodiments, the first layer functions as a mesenchymal mitigation layer and the second layer functions as a cell impermeable layer. Herein, the term “first layer” is used interchangeably with “mesenchymal mitigation layer” and the term “second layer” is used interchangeably with “cell impermeable layer”. An additional layer(s) may be present on the cell impermeable layer (second layer) on a side opposing the first layer (mesenchymal mitigation layer). In some embodiments, a third layer may permit vascular penetration from the host into the third layer for rapid anchoring and attachment of the implantable membrane construct within the tissue of the host. As such, the third layer may be used interchangeably herein with “vascularization layer”. The second, cell impermeable layer is positioned within the interior of the implantable membrane construct, such as, for example, between the first layer and the third layer. The implantable membrane construct has a maximum pore size (MPS) that is less than about 2.0 microns. The first layer and the third layer each has a externally facing side with a surface roughness greater than about 0.5 microns. In embodiments that include three layers, the surface roughness (Sa) of each opposing side of the membrane construct is defined by the externally facing side of the first layer and the externally facing side of the third layer. In embodiments that include two layers, the surface roughness (Sa) of each opposing side of the membrane construct is defined by the externally facing side of, for example, the first layer and the externally facing side of the second layer. The externally facing sides have a surface roughness greater than about 0.5 microns. The first layer, the second layer, and the third layer are bonded or otherwise connected to each other. The implantable membrane construct may include a single layer, two layers, three layers, or more. As described herein, no matter how many layer(s) are included in the implantable membrane construct, the externally facing sides of the implantable membrane construct will each have a surface roughness of at least 0.5 microns. It is to be appreciated that the term “about” as used herein denotes +/−10% of the designated unit of measure.

Further described herein are devices for encapsulating biological entities (e.g., cells), where the encapsulation devices are implanted into a patient, such as into a tissue bed, to provide a biological therapy. It is to be appreciated, however, that embodiments described herein may be applied to a wide variety of implantable medical devices and an encapsulation device as described herein is meant only to be exemplary in nature.

Biological entities suitable for use with the implantable membrane construct and implantable membrane include, but are not limited to, cells, viruses, viral vectors, bacteria, proteins, polysaccharides, and antibodies. It is to be appreciated that if a biological entity other than a cell is selected for use herein, the bioactive component or product of the biological entity needs to be able to pass through the second layer, but not the entity itself. For simplicity, herein the biological entity is referred to as a cell, but nothing in this description limits the biological entity to cells or to any particular type of cell, and the following description applies also to biological entities that are not cells.

Various types of prokaryotic cells, eukaryotic cells, mammalian cells, non-mammalian cells, and/or stem cells may be used with the implantable membrane construct or implantable membrane described herein. In some embodiments, the cells secrete a therapeutically useful substance. Such therapeutically useful substances include hormones, growth factors, trophic factors, neurotransmitters, lymphokines, antibodies, or other cell products which provide a therapeutic benefit to the device recipient. Examples of such therapeutic cell products include, but are not limited to, insulin and other pancreatic hormones, growth factors, interleukins, parathyroid hormone, erythropoietin, transferrin, collagen, elastin, tropoelastin, exosomes, vesicles, genetic fragments, and Factor VIII. Non-limiting examples of suitable growth factors include vascular endothelial growth factor, platelet-derived growth factor, platelet-activating factor, transforming growth factors bone morphogenetic protein, activin, inhibin, fibroblast growth factors, granulocyte-colony stimulating factor, granulocyte-macrophage colony stimulating factor, glial cell line-derived neurotrophic factor, growth differentiation factor-9, epidermal growth factor, and combinations thereof. It is to be appreciated that throughout this disclosure the terms “cell” or “cells'” could be replaced by “biological entity” or “biological entities”, respectively. In addition, the terms “mesenchymal cells” and “stromal cells” may be used interchangeably herein.

The first side of the implantable membrane construct creates a suitable environment to minimize, reduce, inhibit, or even prevent the formation of non-target cell types (e.g., mesenchyme derived cells, including but not limited to fibroblasts, endothelial cells, macrophages) while allowing the encapsulated cells access to oxygen and cell-sustaining nutrients. The presence of mesenchymal cells, particularly at the luminal interface, is undesirable because these non-functioning cells may consume oxygen that could otherwise be utilized by a functioning cell population. In addition, the mesenchymal cells take up space in a lumen that could otherwise be utilized by a functioning cell population. Further, the mesenchymal cells undesirably increase the diffusional barrier distance between the host and a functioning cell population. By designing the membrane construct such that the side adjacent to the lumen in an encapsulation device has a minimum surface roughness, the formation of mesenchymal cells at the luminal interface is reduced, or even prevented, thereby allowing a maximized mass transport of oxygen and nutrients to the graft cell population and permitting the graft cell population to achieve its highest therapeutic potential. As shown in the Examples, when the luminal surface of the implantable membrane construct has a surface roughness (Sa) greater than about 0.5 microns, mesenchymal cells do not form at the interface of the lumen and the first side (i.e., luminal interface) such that the mesenchymal cells do not impede the flow of oxygen and nutrients to the graft cells. It is to be noted that mesenchymal cells may individually be located at the luminal interface, but they do not spread, expand, or impede or prevent the ingress of oxygen and nutrients needed for growth of the encapsulated cells. Additionally, in at least one embodiment, thickness of the first layer (i.e., mesenchymal mitigation layer) may range from about 2 microns to about 100 microns, from about 2 microns to about 90 microns, from about 2 microns to about 80 microns, from about 2 microns to about 70 microns, from about 3 microns to about 60 microns, from about 3 microns to about 50 microns, from about 3 microns to about 45 microns, from about 5 microns to about 40 microns, from about 5 microns to about 35 microns, from about 5 microns to about 30 microns, from about 5 microns to about 35 microns, or from about 5 microns to about 30 microns.

As discussed above, the implantable membrane construct has a maximum pore size (MPS) less than about 2 microns and opposing sides that have a surface roughness (Sa) that is greater than about 0.5 microns. Each opposing side has a micro-roughened surface. As used herein, the term “micro-roughened surface” is meant to denote a surface roughness that is greater than about 0.5 microns but less than about 30 microns. In some embodiments, the surface roughness may be from about 0.5 microns to about 25 microns, from about 0.5 microns to about 20 microns, from about 0.5 microns to about 15 microns, from about 0.5 microns to about 15 microns, from about 0.5 microns to about 10 microns, or from about 0.5 microns to about 5 microns. This is in contrast to “macro-roughened surface” which denotes a surface roughness that is visible to the naked eye. In addition, the surface roughness (Sa) on one side of the implantable membrane construct may be the same as or different from the surface roughness (Sa) on the opposing side of the implantable membrane construct. Although the external, exposed surfaces of the implantable membrane construct each have a certain surface roughness, it is to be noted that the outer layers of the implantable membrane construct may naturally be rough or uneven with a natural surface roughness that is greater than about 0.5 microns. In certain embodiments, however, the surface roughness of the opposing sides of the implantable membrane construct may be accomplished by plasma etching, laser etching, micro-embossing, plasma treatment, 3D printing, deposition, mechanical roughening, or a combination thereof.

In embodiments where the first layer is the sole layer in the implantable membrane construct and in embodiments where there are multiple layers, the implantable membrane construct has a maximum pore size MPS of less than about 2.0 microns. The MPS may range from about 0.05 micron to about 2 microns, from about 0.05 micron to about 1.75 microns, from about 0.05 micron to about 1.5 microns, from about 0.05 micron to about 1.0 micron, from about 0.05 micron to about 0.75 microns, from about 0.05 micron to about 0.5 microns, or from about 0.1 micron to about 1 micron, from about 0.1 to about 0.75 microns, or from about 0.1 micron to 0.5 microns as measured by porometry.

In addition, opposing sides of the implantable membrane construct have a surface roughness greater than about 0.5 microns, greater than about 1 micron, greater than about 5 microns, greater than about 10 microns, greater than about 15 microns, greater than about 20 microns, or greater than about 25 microns. In some embodiments, the surface roughness of one or both of the opposing sides of the implantable membrane construct is from about 0.5 microns to about 25 microns, from about 0.5 microns to about 20 microns, from about 0.5 microns to about 15 microns, from about 0.5 microns to about 15 microns, from about 0.5 microns to about 10 microns, or from about 0.5 microns to about 5 microns. Additionally, the surface roughness along the opposing sides may be substantially homogenous. In other words, the surface roughness (Sa) as measured by the coefficient of variation across the samples taken across various locations where the sample area is no greater than about 0.1 mm² is less than 15%.

In multilayer embodiments, the first layer of the implantable membrane construct includes at least one side that has a surface roughness (Sa) greater than about 0.5 microns. When utilized in an implantable medical device, a side having a surface roughness greater than about 0.5 microns faces the lumen (i.e., luminal interface) and functions as a mesenchymal mitigation layer.

The implantable membrane construct may include a second layer positioned on the first layer. The second layer may be a cell impermeable layer that serves as a microporous cell isolation barrier, is impervious to vascular ingrowth, and prevents cellular contact from the host. The second layer maintains sufficient porosity to allow the passage of molecules (i.e., cellular nutrients, oxygen, waste products, and therapeutic substances) therethrough. The second layer may be considered a “tight” layer in that it restricts or prevents vascular and/or cellular ingrowth as well as cellular contact between the graft cells and the host cells. As discussed previously, the membrane construct may be defined by the externally facing side of the first layer and the externally facing side of the second layer.

Further, the implantable membrane construct may also include a third layer. The third layer is positioned on the second layer on a side opposing the first layer. The externally facing side of the third layer has a surface roughness (Sa) greater than about 0.5 microns. The third layer may be a vascularization layer that permits vascular penetration from the host to allow rapid anchoring and attachment of the implantable membrane construct within the tissue of the host. The vascularization layer is considered to be an “open” layer. In this regard, the vascularization layer may be designed such that there is feature spacing (e.g., space between neighboring features) to enable host integration and attachment. The nodal feature spacing has increased pore sizes to facilitate a more rapid ingrowth of the tissue into the vascularization layer (i.e., third layer). It is to be noted that the first side of the membrane construct may be defined by the first layer and the second side of the membrane construct may be defined by the third layer. The average feature spacing (i.e., space between neighboring features) may be greater than about 3 microns, greater than about 5 microns, greater than about 10 microns, greater than about 25 microns, greater than about 50 microns, greater than about 75 microns, greater than about 100 microns, greater than about 125 microns, greater than about 150 microns, greater than about 175 microns, greater than about 200 microns, greater than about 225 microns, greater than about 250 microns, or greater than about 275 microns. In addition, the average feature spacing may be from about 3 microns to about 300 microns, from about 3 microns to about 275 microns, from about 3 microns to about 250 microns, from about 3 microns to about 225 microns, from about 3 microns to about 200 microns, from about 10 microns to about 175 microns, from about 10 microns to about 150 microns, from about 10 microns to about 125 microns, from about 10 microns to 100 microns, or from about 10 microns to about 50 microns. In some embodiments, the third layer may include biocompatible textiles including woven and non-woven fabrics (e.g., spunbound non-wovens, melt blown fibrous materials, electrospun nanofibers, etc.), non-fluoropolymer membranes such as, nanofibers, polysulfones, polyethersulfones, polyarylsulfones, polyether ether ketone (PEEK), polyethylenes, polypropylenes, and polyimides.

It is to be appreciated that additional layer(s) may additionally or alternatively be present in the implantable membrane construct. For instance, a reinforcing component may be provided to the implantable membrane construct to minimize distortion in vivo so that the cell bed thickness is maintained (e.g., in an encapsulated device). This additional, optional reinforcing component provides a stiffness to the implantable membrane construct that is greater than the implantable membrane construct itself to provide mechanical support. This optional reinforcing component could be continuous in nature, or it may be present in discrete layers on the implantable membrane construct, e.g., patterned across the entire surface of implantable membrane construct or located in specific locations such as around the perimeter of the implantable membrane construct (e.g., a frame). Non-limiting patterns suitable for the reinforcing component on the surface of the implantable membrane construct include dots, straight lines, angled lines, curved lines, dotted lines, grids, etc. The patterns forming the reinforcing component may be used singly or in combination. In addition, the reinforcing component may be temporary in nature (e.g., formed of a bioabsorbable material) or permanent in nature (e.g., a polyethylene terephthalate (PET) mesh or Nitinol). As is understood by one of ordinary skill in the art, the impact of component stiffness depends not just on the stiffness of a single component, but also on the location and restraint of the reinforcing component in the final device form. In order for a component (e.g., a reinforcing component) to be practically useful for adding stiffness to the implantable membrane construct, the reinforcement component should have a stiffness greater than about 0.01 N/cm, although a final determination of the stiffness needed will depend on location and restraint in the finished cell encapsulation device. In some embodiments, the reinforcement component may have a stiffness from about 0.01 N/cm to about 5 N/cm, from about 0.05 N/cm to about 4 N/cm, from about 0.1 N/cm to about 3 N/cm, or from about 0.3 N/cm to about 2 N/cm.

In at least one embodiment, the reinforcing component may be provided on the external surface of the outermost layer (e.g., vascularization layer) to strengthen the implantable membrane construct against environmental forces. In this orientation, the reinforcing component has a pore size sufficient to permit vascular ingrowth. Materials useful as the reinforcing component include materials that are significantly stiffer than the implantable membrane construct. Such materials include, but are not limited to, open mesh biomaterial textiles, woven textiles, non-woven textiles (e.g., collections of fibers or yarns), and fibrous matrices, either alone or in combination. In another embodiment, patterned grids, screens, strands, or rods may be used as the reinforcing component. Additionally, the reinforcing component may be oriented within or between the first, second, and third layers at discrete layers or within the composite layers themselves. It is to be appreciated that the reinforcing component could be located externally, internally (e.g., between the layers), within the layer(s) of the implantable membrane construct, or combinations thereof.

Alternatively, or in addition to the reinforcing component, a frame may surround the implantable membrane construct to improve handleability and stiffness. In some embodiments, the frame is positioned around the perimeter of the implantable membrane construct. The material the frame can be constructed from is not particularly limited as long as it fills the basic needs of being compatible with the implant environment and having the necessary stiffness. Polymer materials such as polyetheretherketone (PEEK), polyethylene terephthalate (PET), polypropylene, polyethylene, polymethyl methacrylate, polyethyl methacrylate, polyacrylate, poly-alpha-hydroxy acids, polycaprolactones, polydioxanones, polyesters, polyglycolic acid, polyglycols, polylactides, polyorthoesters, polyphosphates, polyoxaesters, polyphosphoesters, polyphosphonates, polysaccharides, polytyrosine carbonates, silicones, polyurethanes, polyurethanes with ionic or mesogenic components made by a pre-polymer method, a block copolymer of polyethylene terephthalate (PET) and polyethyleneoxide (PEO), block copolymers containing polystyrene and poly(1,4-butadiene), and an ABA triblock copolymer made from poly(2-methyl-2-oxazoline) and polytetrahydrofuran, and copolymers or polymer blends thereof. Metallic frames can also be incorporated using materials such as spring tempered 316 SST; a spring-tempered cobalt-chromium alloy, such as Co-28Cr-6Mo or Co-35Ni-20Cr-10Mo; a spring-tempered titanium based alloy, such as Ti-6Al-4V or a spring-tempered nickel-titanium alloy, such as Nitinol or copper-aluminum-nickel, copper-zinc-aluminum, and iron-manganese-silicon alloys. The frame materials may be a material that is inherently biocompatible or may be a material that lacks inherent biocompatibility but is rendered biocompatible, such as with a biocompatible coating. Non-limiting examples of inherently biocompatible frame materials include PEEK, Nitinol or Ti-6Al-4V. Non-limiting examples of materials that could be used as a biocompatible coating include PTFE, FEP, and parylene. Solvent-based fluoropolymers may also be useful as biocompatible coatings.

In some embodiments, the implantable membrane construct is perforated to allow direct contact of host cells and vasculature with graft cells contained within a lumen of an encapsulation device. The perforation size, number and location can be selected to optimize graft function and therapeutic potential. The perforations may be of sufficient size to allow host vascular tissue (such as capillaries) to enter the device lumen in order to support the contained biological entities. Perforations allowing for vascular structures to grow into the device lumen help long term health and function of the graft cells because they become directly perfused by the host circulatory system. While some host tissue penetration of the device will improve the therapeutic function of the biological moieties (such as vascular structures and capillaries), other host tissue populations are detrimental to the encapsulated biological moieties by taking up space and volume in the lumen that could otherwise be used therapeutically functional entities. Because perforations also allow host immune cell contact with graft cells, the graft cells are no longer protected from immune rejection unless the host is immunocompromised, treated with immunosuppressant drugs, or the grafted cells are hypoimmunogenic or immunologically matched with the host.

In a perforated device embodiment, the membrane construct described can also prevent the presence and magnitude of certain detrimental host cell populations that are able to enter, spready and expand within the lumen of the device. These host cell populations can also take away valuable space in the lumen for functional, therapeutic cells. One such host cell population could be fibroblasts. Another host population could be other mesenchymal cells, and/or any other cells that may reduce the overall function or available volume for the therapeutic cells.

The implantable membrane construct has a total thickness. “Total thickness” as used herein is meant to denote the thickness of the implantable membrane construct, namely, a total thickness of the layer(s) present in the membrane composite in the z-direction. In some embodiments, the composite thickness may range from about 15 microns to about 200 microns, from about 15 microns to about 175 microns, from about 15 microns to about 150 microns, from about 15 microns to about 125 microns, from about 15 microns to about 100 microns, from about 15 microns to about 75 microns, or from about 15 microns to about 50 microns.

In addition, the implantable membrane construct has a mean flow pore size that is greater than about 0.10 microns. The mean flow pore size may be greater than about 0.5 microns, greater than about 1.0 micron, greater than about 1.5 microns, or greater than about 2.0 microns. Additionally, the mean flow pore size may range from about 0.1 microns to about 3.0 microns, from about 0.1 microns to about 2.75 microns, from about 0.1 microns to about 2.5 microns, from about 0.1 microns to about 2.0 microns, or from about 0.1 microns to about 1.5 microns. The mean flow pore size ensures that the pore size is large enough to allow mass transport and diffusion.

It is necessary to balance the tradeoffs of the competing properties of strength and diffusion resistance. Strength is important for handling, the ability to manufacture encapsulation device, and to ensure device integrity in vivo. The tensile strength in the weakest direction of the implantable membrane construct is greater than about 0.15 N/mm, greater than about 0.25 N/mm, greater than about 0.50 N/mm, greater than about 0.75 N/mm, greater than about 1 N/mm, greater than about 1.15 N/mm, or greater than about 1.25 N/mm. Additionally, the maximum tensile strength in the weakest direction may range from about 0.15 N/mm to about 1.5 N/mm, from about 0.15 N/mm to about 1.25 N/mm, from about 0.15 N/mm to about 1 N/mm, from about 0.25 N/mm to about 0.75 N/mm, or from about 0.3 N/mm to about 0.5 N/mm.

The implantable membrane construct also has a z-strength. The z-strength is sufficient to prevent delamination of the membrane construct in vivo. In some embodiments, the layers are intimately bonded or otherwise connected to each other to form the implantable membrane construct. As used herein, “intimately bonded” refers to layers of the implantable membrane construct that are not readily separable or detachable at any point on their surface. In a two-layer construct, the first and second layers are intimately bonded. In a third (or more) layer construct, at least two of the first, second, and third layers are intimately bonded.

The porosity of the implantable membrane construct is one that is sufficient to allow the flowthrough of cell nutrients, cell waste, and therapeutic substances but no sufficient to pass cells therethrough. In certain embodiments, the porosity of the implantable membrane construct is greater than about 50%, greater than about 60%, greater than about 70%, or greater than about 80%. Additionally, the porosity of the implantable membrane construct may range from about 50% to about 98%, from about 50% to about 90%, from about 50% to about 80%, or from about 60% to about 90%.

The implantable membrane composite may have at least partially thereon a surface coating, such as a Zwitterion non-fouling coating, a hydrophilic coating, or a CBAS®/Heparin coating (commercially available from W.L. Gore & Associates, Inc.). The surface coating may also or alternatively contain antimicrobial agents, antibodies (e.g., anti-CD 47 antibodies (anti-fibrotic)), pharmaceuticals, and other biologically active molecules (e.g., stimulators of vascularization such as FGF, VEGF, endoglin, PDGF, angiopoetins, and integrins; Anti-fibrotic such as TGFb inhibitors, sirolimus, CSFIR inhibitors, and anti CD 47 antibody; anti-inflammatory/immune modulators such as CXCL12, and corticosteroids), and combinations thereof.

In at least one embodiment, the layers (e.g., the first layer and the second layer or the first layer, second layer, and third layer) are bonded together by one or more biocompatible adhesive to form the implantable membrane construct. The adhesive may be applied to the surface of one or more of the layers to create a bond between the layers. Non-limiting examples of suitable biocompatible adhesives include fluorinated ethylene propylene (FEP), Bionate® (a polycarbonate urethane), a polycarbonate urethane, a thermoplastic fluoropolymer comprised of TFE and PAVE, EFEP (ethylene fluorinated ethylene propylene), PEBAX (a polyether amide), PVDF (poly vinylidene fluoride), Carb-O-Sil® (an absilicone polycarbonate urethane), Elasthane™ (a polyether urethane), PurSil® (a silicone polyether urethane), polyethylene, high density polyethylene (HDPE), ethylene chlorotetrafluoroethylene (ECTFE), perfluoroalkoxy (PFA), polypropylene, polyethylene, polyethylene terephthalate (PET), and combinations thereof.

The membrane construct (e.g., one or more of the first layer, the second layer, and the third layer) includes at least a fluoropolymer membrane such as expanded polytetrafluoroethylene, a modified ePTFE membrane, a tetrafluoroethylene (TFE) copolymer membrane, a polyvinylidene fluoride (PVDF) membrane, or a fluorinated ethylene propylene (FEP) membrane. Expanded polytetrafluoroethylene (ePTFE) (and other fibrillated polymers) has a node and fibril microstructure where the nodes are interconnected by the fibrils and the pores are the space located between the nodes and fibrils throughout the membrane. As used herein, the term “node” is mean to denote a feature consisting of largely polymer material. Expanded polytetrafluoroethylene membranes such as, but not limited to, those prepared in accordance with the methods described in U.S. Pat. No. 3,953,566 to Gore, U.S. Pat. No. 7,306,729 to Bacino et al., U.S. Pat. No. 5,476,589 to Bacino, WO 94/13469 to Bacino, U.S. Pat. No. 5,814,405 to Branca et al. or U.S. Pat. No. 5,183,545 to Branca et al. may be used herein. In some embodiments, the membrane construct may include a non-fluoropolymer membrane such as, but not limited to, a polyethylene membrane.

In some embodiments, at least one of the first layer, second layer, third layer or reinforcing component is formed of a non-woven fabric. There are numerous types of non-woven fabrics, each or which may vary in tightness of the weave and the thickness of the sheet. The non-woven fabric may be a bonded fabric, a formed fabric, or an engineered fabric that is manufactured by processes other than weaving or knitting. In some embodiments, the non-woven fabric is a porous, textile-like material, usually in flat sheet form, formed primarily or entirely of fibers, such as staple fibers assembled in a web, sheet, or batt. The structure of the non-woven fabric is based on the arrangement of, for example, staple fibers that are typically arranged more or less randomly. In addition, non-woven fabrics can be created by a variety of techniques known in the textile industry. Various methods may create carded, wet laid, melt blown, spunbonded, electrospun, or air laid non-woven materials. Exemplary methods and substrates are described, for example, in U.S. Patent Publication No. 2010/0151575 to Colter, et al. In some embodiments, the non-woven fabrics are biocompatible and/or bioabsorbable.

In some embodiments, it may be desirable for one or more of the third layer (i.e., vascularization layer) and/or the reinforcing component to be non-permeant (e.g., biodegradable). In such an instance, a biodegradable material may be used to form the third layer and/or the reinforcing component. Suitable examples of biodegradable materials include, but are not limited to, polyglycolide:trimethylene carbonate (PGA:TMC), polyalphahydroxy acid such as polylactic acid, polyglycolic acid, poly (glycolide), and poly(lactide-co-caprolactone), poly(caprolactone), poly(carbonates), poly(dioxanone), poly (hydroxybutyrates), poly(hydroxyvalerates), poly (hydroxybutyrates-co-valerates), expanded polyparaxylylene (ePLLA), such as is taught in U.S. Pat. No. 9,732,184 to Sbriglia, and copolymers and blends thereof. Alternatively, the third layer may be coated with a bio-absorbable material, or a bio-absorbable material may be incorporated into or onto the third layer in the form of a powder. Coated materials may promote infection site reduction, vascularization, and favorable type 1 collagen deposition.

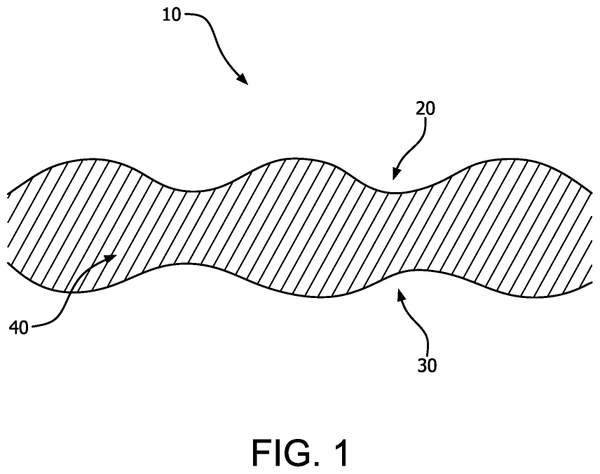

The simplest construct of an implantable membrane construct 10 having a maximum pore size (MPS) less than about 2 microns and two opposing sides 20, 30 each having a surface roughness of at least 0.5 microns is depicted in FIG. 1. The implantable membrane construct 10 is formed of a single layer 40 that operates to mitigate the formation of mesenchymal cells at a luminal interface when placed against the lumen in a cell encapsulation device. In some embodiments, the membrane construct is formed of a fluoropolymer membrane such as, but not limited to, an expanded polytetrafluoroethylene membrane.

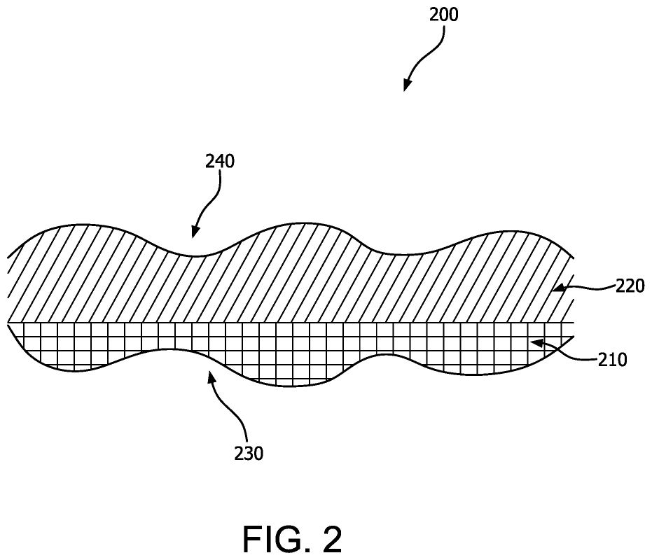

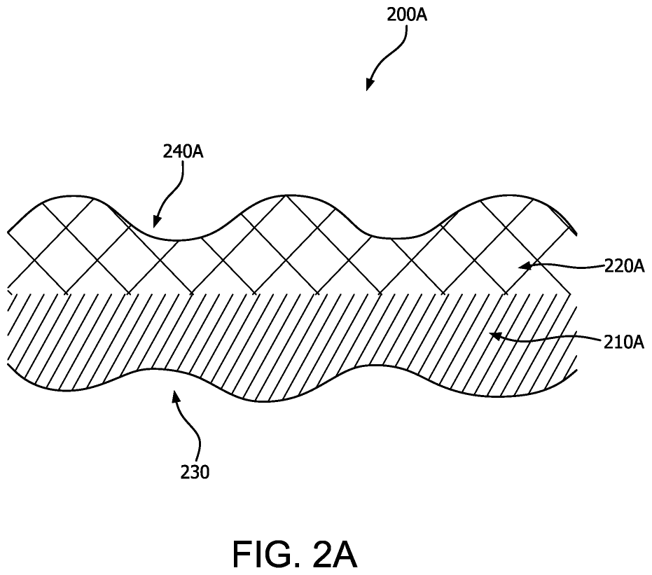

Turning to FIG. 2, a two-layer implantable membrane construct 200 is shown. The implantable membrane construct 200 includes a first layer 210 and a second layer 220 positioned on the first layer 210. When the implantable membrane construct is positioned in a cell encapsulation device, the side of the first layer opposing the side of the second layer (i.e., side 230) has a surface roughness (Sa) of at least about 0.5 microns and is located next to the lumen to form a luminal interface. The first layer 210 and the second layer 220 are adhered or otherwise affixed to each other. It is to be appreciated that, like the embodiment depicted in FIG. 1, the outwardly facing sides 230, 240 each have a surface roughness of at least 0.5 microns and the implantable membrane construct 100 has a maximum pore size (MPS) that is less than about 2 microns.

FIG. 2A depicts another two-layer embodiment of an implantable membrane construct 200A. As depicted, the implantable membrane construct 200A includes first layer 210A (which in some embodiments may be the same as the single layer membrane depicted in FIG. 1) and a second layer 220A. The two sides 210A, 220A may be adhered or otherwise affixed to each other. In the embodiment depicted in FIG. 2A, the external surfaces 230A, 240A of the construct 200A each have a surface roughness of at least 0.5 microns. The first layer 210A provides the function of a mesenchymal mitigation layer and the second layer 220A provides the function of vascularization. The implantable membrane construct 200A has a maximum pore size (MPS) that is less than about 2 microns. When the implantable membrane construct 300 is positioned in a cell encapsulation device, the side of the first layer 210A is located next to the lumen to form a luminal interface.

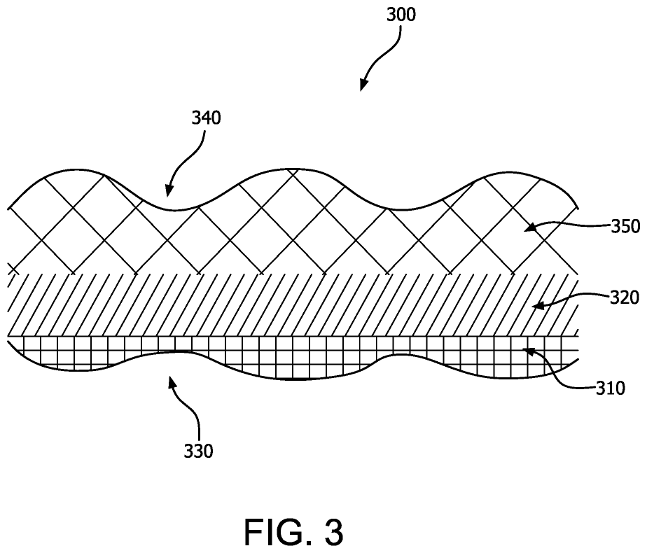

FIG. 3 depicts an embodiment where the implantable membrane construct 300 has a first layer 310, a second layer 320, and a third layer 350. The third layer 350 is positioned on a side of the second layer 320 opposing the first layer 310. In other words, the second layer 320 is positioned between the first layer 310 and the third layer 350. As described herein, the first layer 310 is a mesenchymal mitigation layer (e.g., open layer), the second layer 320 is a cell impermeable layer (e.g., a tight layer), and the third layer 350 is a vascularization layer (e.g., an open layer). When the implantable membrane construct 300 is within a cell encapsulation device, the first layer 310 is positioned on the lumen to form a luminal interface. The first side and the second side of the implantable membrane construct 400 has a surface roughness (Sa) greater than about 0.5 microns. Additionally, the implantable membrane construct 300 has a maximum pore size that is less than about 2.0 microns.

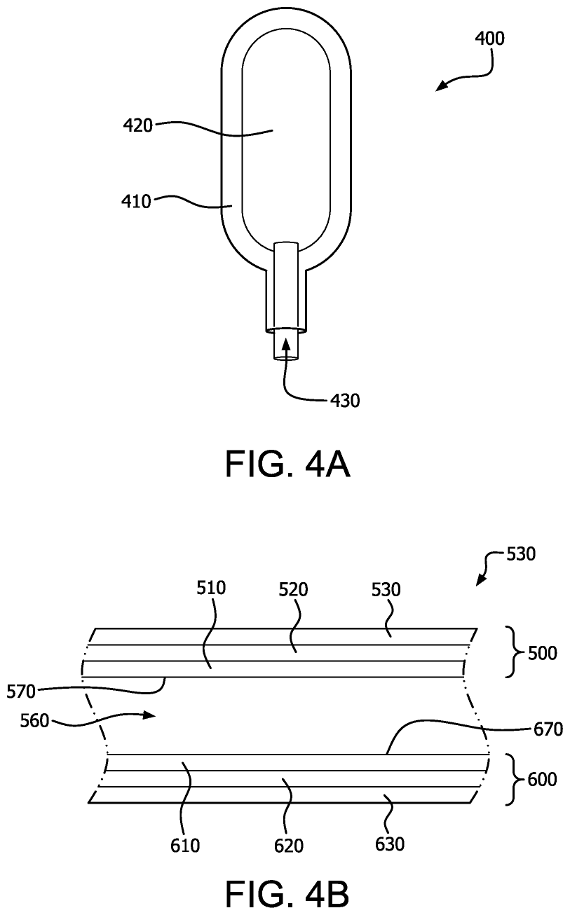

The implantable membrane construct can be manufactured into various forms including, but not limited to, a housing, a chamber, a pouch, a tube, or a cover. In some embodiments, a cell encapsulation device may be implemented for providing therapeutic substances to an individual in need of treatment. In one embodiment, the implantable membrane construct forms a cell encapsulating device as illustrated in FIG. 4A. FIG. 4A is a top view of an exemplary cell encapsulating device 400 formed with two implantable membrane constructs. The two implantable membrane constructs are sealed along at least a portion of their periphery 410. Only the externally facing layer of one of the implantable membrane constructs 420 is shown in FIG. 4A. The cell encapsulating device 400 includes an internal chamber (not shown) for containing cells therein and a port 430 that extends into the internal chamber and is in fluid communication therewith.

FIG. 4B is a cross-sectional illustration of the cell encapsulation device of FIG. 4A. As shown, a first implantable membrane construct 500 having a first layer 510, a second layer 520, and a third layer 550 is positioned adjacent to a second implantable membrane construct 600 that has a first layer 610, a second layer 620, and a third layer 630. The first layers 510, 610 of the implantable membrane constructs 500, 600, respectively, have a surface roughness (Sa) greater than about 0.5 microns that are positioned next to the lumen 560 to form luminal interfaces 570, 670. The externally facing side of the third layers 530, 630 also have a surface roughness (Sa) greater than about 0.5 microns. An optional reinforcing component is not depicted in FIG. 4B, although it could be utilized in this embodiment. The lumen 560 is located between the two membrane composites 500, 600 for the placement and retention of cells (and/or other biological entities).

The cell encapsulation devices are capable of explantation or removal from the patient such as if the patient goes into remission and no longer needs the device or the device needs to be taken out for other reasons such as implant expiration or a severe immunologic response. In such a case, a new encapsulation device may be implanted.

Having generally described this disclosure, a further understanding can be obtained by reference to certain specific examples illustrated below which are provided for purposes of illustration only and are not intended to be all inclusive or limiting unless otherwise specified,

### Test Methods

### Surface Roughness (Sa)

Surface roughness was measured using a Keyence VK-X1000 Laser Scanning Confocal Microscope and associated Multi File Analyzer Software. Unless otherwise noted, materials were tested after the application of any coatings. Samples were immobilized to a 5 mm metal stub, using a carbon tape adhesive. The side opposite the side intended for imaging was laid on top of the adhesive and the sample was then cut to the stub diameter. Images were then taken using the VK-X1000 at magnifications between 20× and 150×. Surface areas from 0.01-0.1 mm² were used in analysis of the roughness of the surfaces. This range allowed for sufficient resolution and representative analysis of the layers.

The image was then processed in the Multi File Analyzer Software to account for any wavy surfaces or large curvatures by performing a surface shape correction. The surface shape correction was performed using a waveform removal at a correction strength of 5 for the height data across the entirety of the image. After the image was preprocessed, the areas to be analyzed and parameters to be calculated were set. The Multi File Analyzer software automatically performs the calculations.

### Maximum Pore Size (MPS)

Maximum pore size was measured per ASTM F316 using a Quantachrome 3 Gzh porometer from Anton Paar and silicone oil (20.1 dyne/cm) as a wetting solution.

### Mean Flow Pore Size

The mean flow pore size test was performed using ASTM F316 as a Standard Test Method.

### Porosity

The porosity of a layer is defined herein as the proportion of layer volume consisting of pore space compared to the total volume of the layer. The porosity is calculated by comparing the bulk density of a porous construct consisting of solid fraction and void fraction to the density of the solid fraction using the following equation:

$$Porosity = ( 1 - \frac{{Density}_{Bulk}}{{Density}_{Solid Fraction}} ) \times 100 \% .$$

### Tensile Strength, Weakest Direction

Materials were tested for maximum tensile load using a 5500 Series Instron® Electromechanical Testing System. Unless otherwise noted, materials were tested after the application of any coatings. Samples were cut oriented in the longitudinal and transverse axes of the material using a laser cutting system to create a sample in the shape of a D412F dogbone. The samples were then loaded into the Instron® tester grips and tested at a constant rate of 20 in/m in until failure. The maximum load sustained during testing was normalized by specimen gauge width (6.35 mm for D412F samples) to define maximum tensile load. The lower of the two results defined the tensile strength in the weakest direction.

### Z-Strength

Materials were tested for composite bond strength using a 5500 Series Instron® Electromechanical Testing System. Unless otherwise noted, materials were testing for tensile strength prior to the application of any coatings. Samples were fixed to a 1″×1″ steel platen using 3M 9500PC double sided tape and loaded into the Instron® with an opposing 1″×1″ steel platen with 3M 9500PC double sided tape on its surface. A characteristic compressive load of 1001 N was applied for 60 s to allow adhesive to partially penetrate the structure. After this bonding, the platens were separated at a constant rate of 20 in/s until failure. The maximum load was normalized by the test area (defined as the 1″×1″ test area) to define the composite bond.

### Thickness

Samples for thickness measurements were cut and placed in a thin bottom imaging petri dish with a glass cover slip on top. The sample was then wet out with DI water and placed on a laser scanning confocal microscope equipped with a Photomultiplier tubes (PMT) detector. The thickness of the sample was acquired by capturing the refractive index differences with a fixed wavelength laser light.

The confocal function of the microscope allows the scanning of the sample structure at different depths. Through stitching of the optical slices (individual image at each z position), the total thickness of the sample, or regions of the sample, is obtained.

### SEM Sample Preparation

SEM samples were prepared by first fixing the membrane composite or membrane composite layer(s) to an adhesive for handling, with the side opposite the side intended for imaging facing the adhesive. The film was then cut to provide an approximately 3 mm×3 mm area for imaging. The sample was then sputter coated using an Emitech K550X sputter coater and platinum target. Images were then taken using a FEI Quanta 400 scanning electron microscope from Thermo Scientific and/or SU-8230 scanning electron microscope from Hitachi at a magnification and resolution that allowed visualization of a sufficient number of features for robust analysis while ensuring each analyzed feature's minimum dimension was at least five pixels in length.

### Feature Spacing

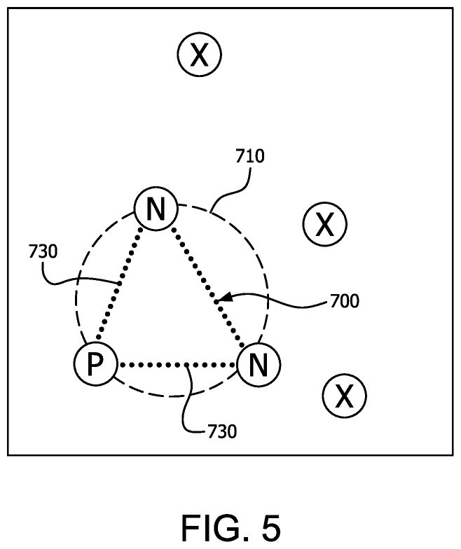

Feature spacing was determined by analyzing SEM images in ImageJ 1.51h from the National Institute of Health (NIH). The image scale was set based on the scale provided by the SEM image. “Features” as used herein may be defined as three dimensional components within the layer that are generally immovable and resistant to deformation when exposed to environmental forces, such as, but not limited to, cell movement (e.g., cellular migration and ingrowth, host vascularization/endothelial blood vessel formation). Features were identified and isolated through a combination of thresholding based on size/shading and/or manual identification. In instances where the structure consists of a continuous structure, such as a nonwoven or etched surface, as opposed to a structure with discrete solid features, solid features are defined as the portion of the structure surrounding voids with the corresponding spacing extending from one side of the void to the opposing side. After isolating the features, a Delaunay Triangulation was performed to identify neighboring features. Triangulations whose circumcircle extended beyond the edge of the image were disregarded from the analysis. Lines were drawn between the nearest edges of neighboring features and measured for length to define spacing between neighboring features (see, e.g., FIG. 5). As shown pictorially in FIG. 5, the designated feature (P) is connected to neighboring features (N) to form a triangle 700 where the circumcircle 710 contains no solid features within. Solid features (X) designate the solid features that are not neighboring features. Thus, in the instance depicted in FIG. 5, the feature spacing 730 is the straight distance between the designated features (P), (N).

### Stiffness

A stiffness test was performed based on ASTM D790-17 Standard Test Method for flexural properties of unreinforced and reinforced plastics and electrical insulating material. This method was used for reinforcing layer and/or the final membrane construct.

Procedure B was followed and includes >5% strain and type 1 crosshead position for deflection. The dimensions of the fixture were adjusted to have a span of 16 mm and a radius of support and nosepiece of 1.6 mm. The test parameters used were a deflection of 3.14 mm and a test speed of 96.8 mm/min. In cases where the sample width differed from the standard 1 cm, the force was normalized to a 1 cm sample width by the linear ratio.

Integration of Implantable Membrane Construct into a Device Form

In order to evaluate the in vivo utility, various implantable membrane constructs were manufactured into a device form suitable for use as an implantable encapsulation device for the delivery of a cell therapy. In this test form, two identical membrane composites were sealed around a perimeter region to form an open internal lumen space accessed by a fill tube or port to enable the loading of cells.

A thermoplastic film acted as the bonding component that created the perimeter seal around the device during the welding operation. The film used was a polycarbonate urethane film. The extruded tube had an outer diameter of 1.60 mm and an inner diameter of 0.889 mm.

Additionally, a reinforcing mechanical support having a suitable stiffness was added to the exterior of the encapsulation device. In particular, a polyester monofilament woven mesh with 120 microns fibers spaced approximately 300 microns from each other was positioned on the outside of both composite membranes (i.e., the exterior of the device). The stiffness of this layer was 0.097 N/cm.

All layers were cut to an approximate 22×11 mm oval outer dimension size using a laser cutting table. The film was cut into oval ring profiles with a 2 mm width and placed in an intercalating stack up pattern on both sides of the implantable membrane construct as well as around the mesh (reinforcing layer). This intercalating stack-up pattern of the components allowed for a melted film bond around each of the composite layers as well as the mesh at a perimeter location. The layers of the implantable membrane construct were stacked symmetrically opposing the filling tube such that the cell impermeable tight layer of the implantable membrane construct was facing internally towards the inner lumen.

An integral perimeter seal around the device was formed by using either an ultrasonic welder (Herrmann Ultrasonics) or a thermal staking welder. With both processes, thermal or vibrational energy and force was applied to the layered stack to melt and flow the thermoplastic film above its softening temperature to weld all the layers together. The device was constructed in a two-step welding process where the energy or heat was applied from one side such that the first composite membrane was integrated into one side of the device followed by the second composite membrane onto the opposing side of the device. The final suitability of the weld was assessed by testing the device for integrity using a pressure decay test with a USON Sprint iQ Leak Tester at a test pressure of 5 psi.

### In Vivo Nude Rat Study and Explant Histology

The encapsulation devices were loaded ex vivo with 6-7×106 cells (or about 20 uL) of pancreatic progenitor cells as described in at least the teachings of U.S. Pat. No. 8,278,106 to Martinson, et. al. After being held in media for less than 24-96 hours, two devices were implanted subcutaneously in each male immunodeficient athymic nude rat. The pancreatic progenitor cells were allowed to develop and mature in vivo. At indicated time points post implant, nude rats were euthanized, and devices were explanted. Excess tissue was trimmed away, and devices were placed in neutral buffered 10% formalin for 6-30 hours. Fixed devices were processed for paraffin embedding in a Leica Biosystems ASP300S tissue processor. Processed devices were cut into 4-6 pieces of approximately 5 mm each and embedded together in paraffin blocks. Multiple 3-10-micron cross sections were cut from each block, placed and slides and stained with hematoxylin and eosin (H&E). Images of the slides were captured using a Hamamatsu Nanozoomer 2.0-HT Digital Slide Scanner.

### EXAMPLES

### Comparative Example 1

### Manufacturing of the Membrane

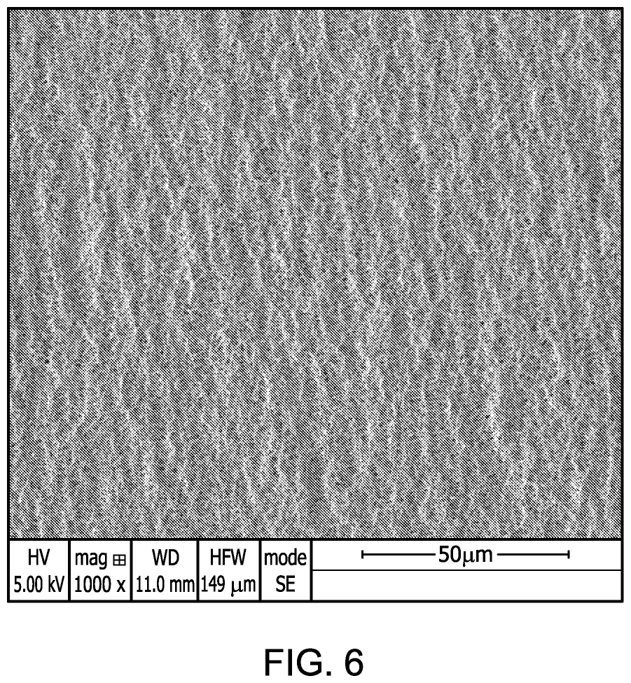

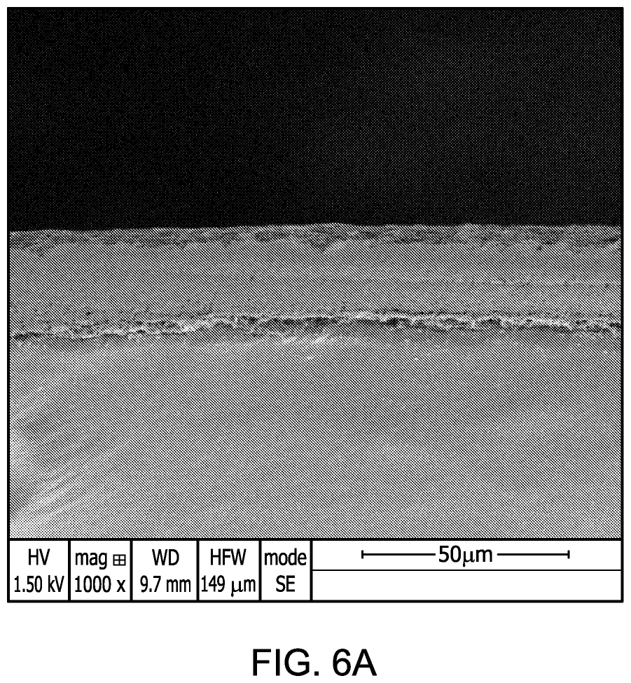

A commercially available, hydrophilic expanded polytetrafluoroethylene (ePTFE) membrane with a Maximum Pore Size (MPS) of 0.43 microns sold under the trade name Biopore® from Millipore (Cork, Ireland) was obtained. This single layer provided a tight, cell impermeable interface while still enabling mass transport of oxygen and nutrients therethrough. The opposing sides of the ePTFE membrane had a surface roughness (Sa) of approximately 0.4 microns each. A representative scanning electron micrograph (SEM) of the surface of this membrane is shown in FIG. 6 and is representative of both sides of this membrane as it is a monolithic single layer membrane. A representative scanning electron micrograph (SEM) of the cross-section of this membrane is shown in FIG. 6A.

### Characterization of the Membrane

The membrane was evaluated and characterized for the relevant parameters necessary for the intended function. Parameters are marked as “N/A” if they are not relevant for that specific function. Parameters are marked as “-” if they are practically unobtainable as a result of how the layers of the construct were processed. The methods used for the characterization of the relevant parameters were performed in accordance with the methods described in “Test Methods” section set forth above. The results of Comparative Example 1 are summarized in Table 1.

TABLE 1

| Relevant Properties: | units | Measured Value |
|---|---|---|
| Surface Roughness (Sa), Lumen-Facing Side | μm | 0.38 |
| Surface Roughness (Sa), Host-Facing Side | μm | 0.38 |
| Maximum Pore Size (MPS) | μm | 0.43 |
| Thickness, Mesenchymal Mitigation Layer | μm | N/A |
| Mean Pore Size | μm | 0.2 |
| Tensile strength | N/mm | 0.4 |
| Total Thickness | μm | 26.6 |
| Z-strength | kPa | N/A |
| Porosity | % | 63.6 |
| Feature Spacing, Lumen-Facing Side | μm | N/A |
| Feature Spacing, Host-Facing Side | μm | N/A |

### Evaluation of the Membrane In Vivo

The ePTFE membrane was ultrasonically welded into a device form in accordance with the Integration of Biocompatible Membrane into a Device Form set forth in the Test Methods section. The device was evaluated in vivo in accordance with the In Vivo Nude Rat Study set forth in the Test Methods section.

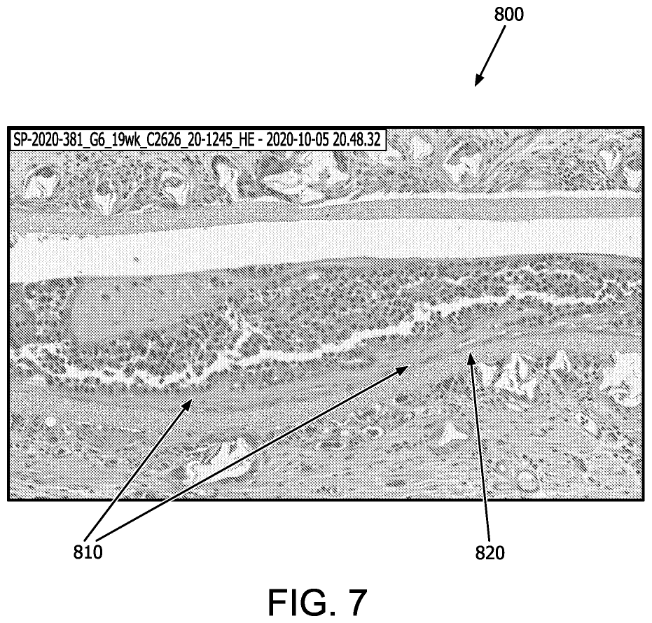

The presence of mesenchymal cells was observed at the cell impermeable layer within the encapsulation device, thereby creating an additional barrier to diffusion and decreasing the functioning cell population within the graft. FIG. 7 illustrates mesenchymal cells 810 lining the luminal interface 820 of the implantable membrane composite 800 at the 19-week timepoint.

### Comparative Example 2

### Manufacturing of the Implantable Membrane Construct

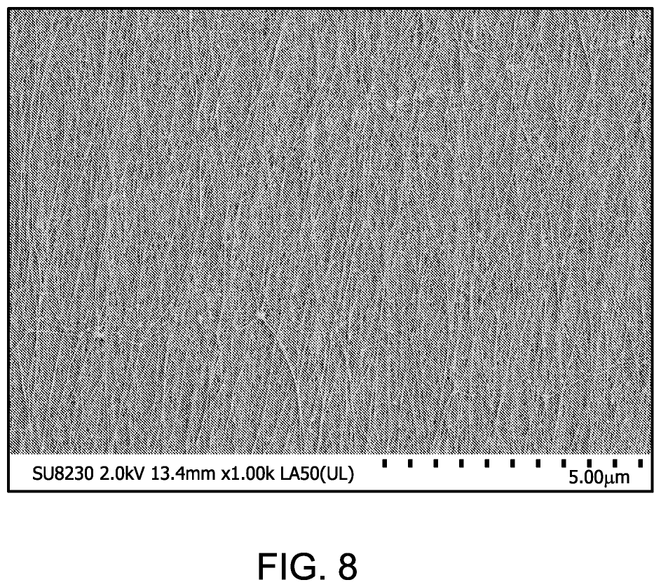

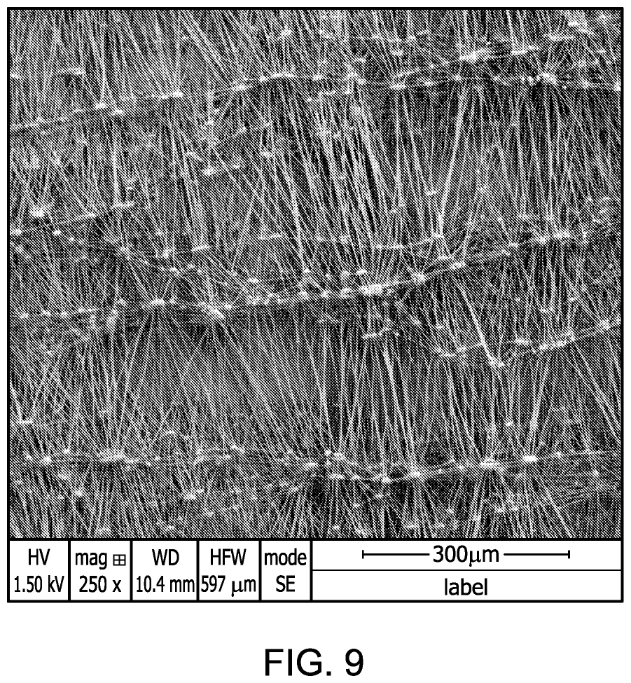

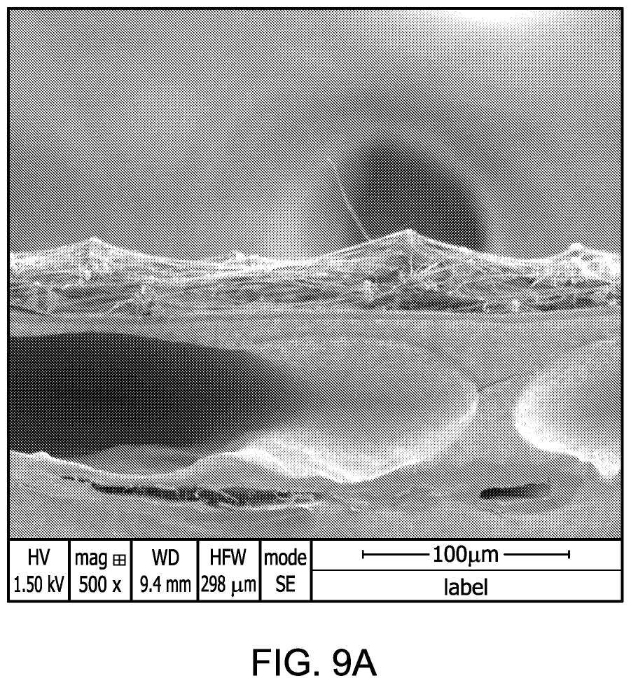

An implantable membrane construct was manufactured in the same manner as the cell encapsulation device described in International Patent Publication WO 2020/243663A1 to Bruhn, et al. and then rendered hydrophilic according to the teachings of U.S. Pat. No. 5,902,745, to Butler, et al. The membrane construct was a two-layer composite comprised of a tight, cell impermeable layer and an open, cell permeable layer. The membrane construct had a maximum pore size (MPS) of 0.41 microns, providing cell impermeability while maintaining transport of oxygen and nutrients therethrough. The open, cell permeable layer of the membrane construct oriented to the exterior, host tissue facing side allowed the ingrowth of tissue and vascularization through to the cell impermeable surface. Representative scanning electron micrographs (SEM) of each side of this membrane construct are shown in FIGS. 8 (lumen facing side) and 9 (host facing side). A representative scanning electron micrograph (SEM) of the cross-section of this membrane construct is shown in FIG. 9A.

### Characterization of the Implantable Membrane Construct

The membrane construct was evaluated and characterized for the relevant parameters necessary for the intended function. Parameters are marked as “N/A” if they are not relevant for that specific function. Parameters are marked as “-” if they are practically unobtainable as a result of how the layers of the construct were processed. The methods used for the characterization of the relevant parameters were performed in accordance with the methods described in “Test Methods” section. The results of Comparative Example 2 are summarized in Table 2.

TABLE 2

| Relevant Properties: | units | Measured Value |
|---|---|---|
| Surface Roughness (Sa), Lumen-Facing Side | μm | 0.41 |
| Surface Roughness (Sa), Host-Facing Side | μm | 3.88 |
| Maximum Pore Size (MPS) | μm | 0.36 |
| Thickness, Mesenchymal Mitigation Layer | μm | N/A |
| Mean Pore Size | μm | 0.18 |
| Tensile strength | N/mm | 0.20 |
| Total Thickness | μm | 23.3 |
| Z-strength | kPa | 307 |
| Porosity | % | 97.0 |
| Feature Spacing, Lumen-Facing Side | μm | N/A |
| Feature Spacing, Host-Facing Side | μm | 33 |

### Evaluation of the Implantable Membrane Construct

The implantable membrane construct was thermally welded into a device form in accordance with the Integration of Biocompatible Membrane into a Device Form as set forth in the Test Methods section and evaluated in vivo. The device was evaluated in accordance with the In Vivo Nude Rat Study set forth in the Test Methods section.

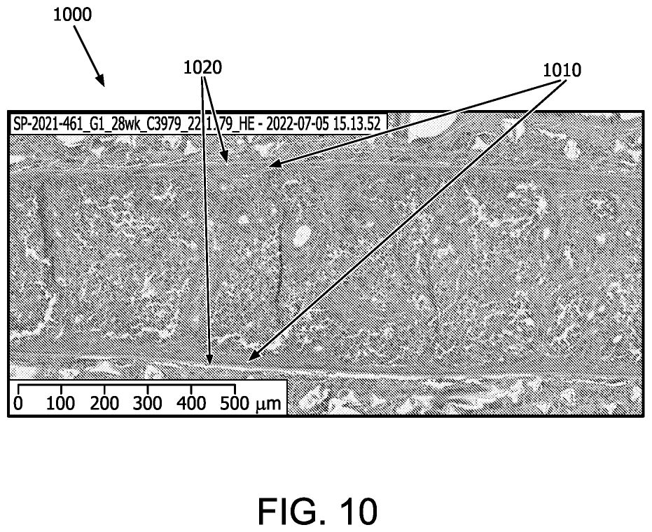

The presence of mesenchymal cells was observed at the cell impermeable layer surface facing the graft cell population. This layer of mesenchymal cells increased the diffusional barrier/distance between the host and the functional cell population and consumed space within the device lumen otherwise available for the functional cell population. This resulted in a decrease of the functional cell population within the graft population. FIG. 10 is a representative histological image that depicts mesenchymal cells 1010 lining the luminal surface 1020 of the implantable membrane construct 1000 at the 28-week timepoint.

### Example 1

### Manufacturing of the Implantable Membrane Construct

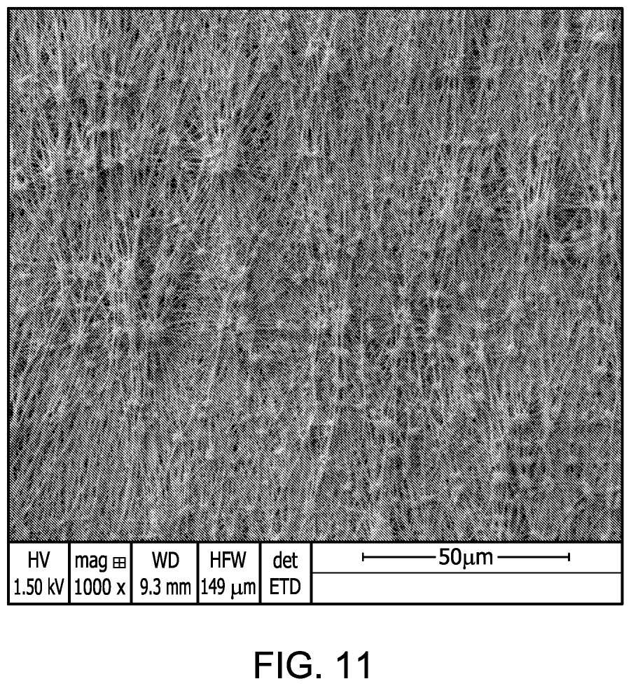

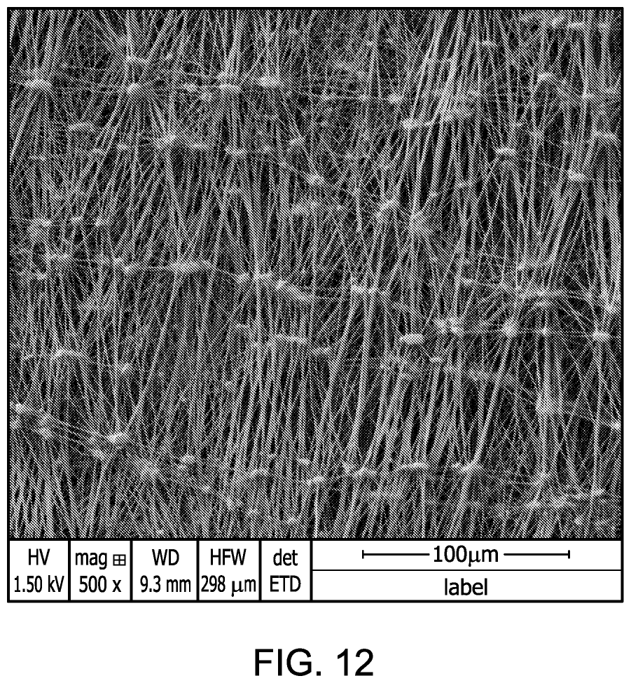

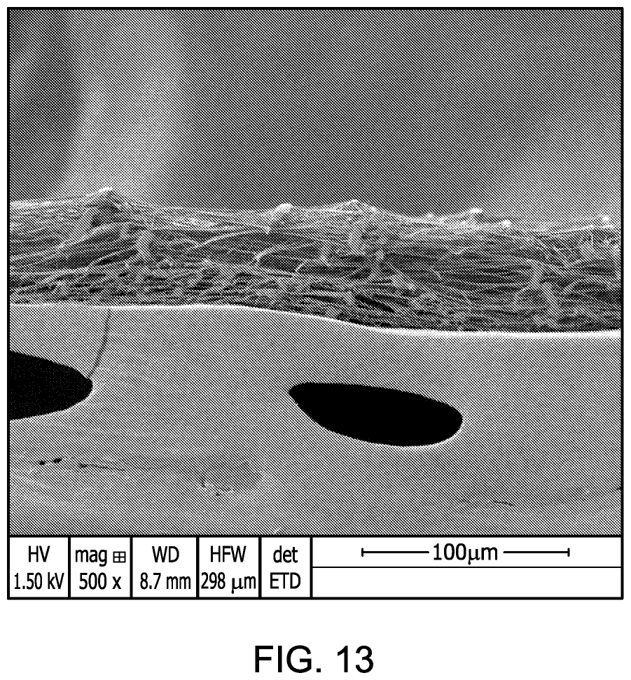

An implantable membrane construct was manufactured in the same manner as the cell encapsulation device described in International Patent Publication WO 2020/243663A1 to Bruhn, et al. with the exception of not adding the third layer. The implantable membrane composite had two layers. One layer (Mesenchymal Mitigation Layer) was an open, cell permeable layer. The other layer (Cell Impermeable Layer) consisted of a tight, cell impermeable layer. The membrane construct had a MPS of 1.78 microns, which provided cell impermeability while maintaining the transport of oxygen and nutrients therethrough. The open layer of the membrane construct was oriented towards the host tissue facing side of the device and contained a cell permeable surface, allowing the ingrowth of tissue and vascularization through to the cell impermeable surface. This host-facing side has a surface roughness of approximately 0.9 microns. The lumen-facing side of this membrane construct was provided by the tight, cell impermeable layer and had a surface roughness of approximately 0.6 microns. Representative scanning electron micrographs (SEM) of the surfaces of this membrane are shown in FIGS. 11 (lumen-facing side) and 12 (host-facing side). A representative scanning electron micrograph (SEM) of the cross-section of this membrane construct is shown in FIG. 13.

### Characterization of the Implantable Membrane Construct

The membrane construct was evaluated and characterized for the relevant parameters necessary for the intended function. Parameters are marked as “N/A” if they are not relevant for that specific function. Parameters are marked as “-” if they are practically unobtainable as a result of how the layers of the construct were processed. The methods used for the characterization of the relevant parameters were performed in accordance with the methods described in “Test Methods” section. The results are summarized in Table 3.

TABLE 3

| Relevant Properties: | units | Measured Value |
|---|---|---|
| Surface Roughness (Sa), Lumen-Facing Side | μm | 0.61 |
| Surface Roughness (Sa), Host-Facing Side | μm | 0.9 |
| Maximum Pore Size (MPS) | μm | 1.78 |
| Thickness, Mesenchymal Mitigation Layer | μm | 20.9 |
| Mean Pore Size | μm | 0.85 |
| Tensile strength | N/mm | 0.15 |
| Total Thickness | μm | 57 |
| Z-strength | kPa | 333 |
| Porosity | % | 94.8 |
| Feature Spacing, Lumen-Facing Side | μm | 4.8 |
| Feature Spacing, Host-Facing Side | μm | 20.2 |

### Evaluation of the Implantable Membrane Construct

The implantable membrane construct was thermally welded into a device form in accordance with the Integration of Biocompatible Membrane into a Device Form as set forth in the Test Methods section. The device was evaluated in vivo in accordance with the In Vivo Nude Rat Study set forth in the Test Methods section.

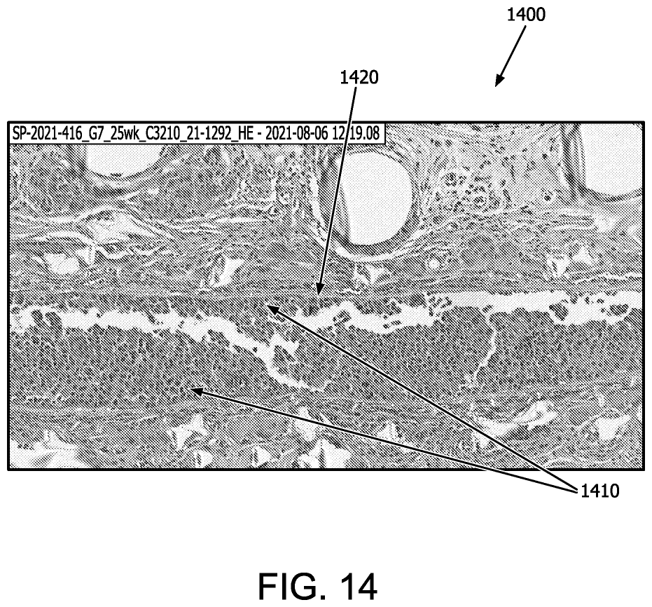

The presence of mesenchymal cells was not observed at the cell impermeable layer surface facing the graft cell population (i.e., the luminal interface). FIG. 14 is a representative histological image that illustrates viable functional cells 1410 adjacent to the luminal surface 1420 of the implantable membrane construct 1400 at the 25-week timepoint.

### Example 2

### Manufacturing of the Implantable Membrane Construct

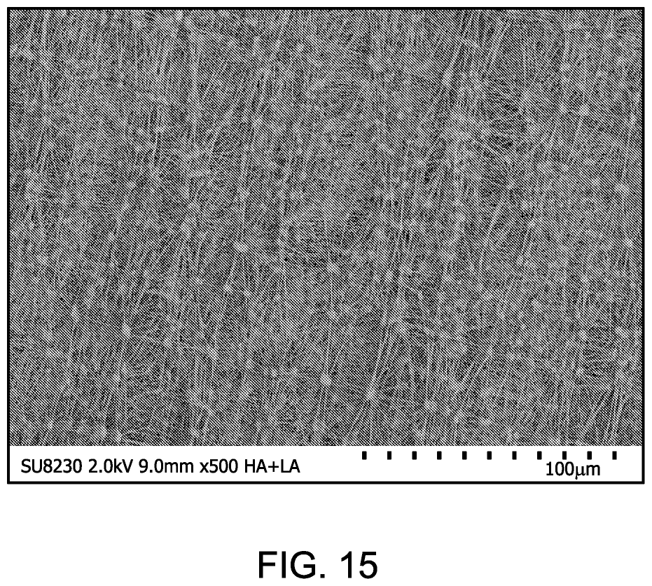

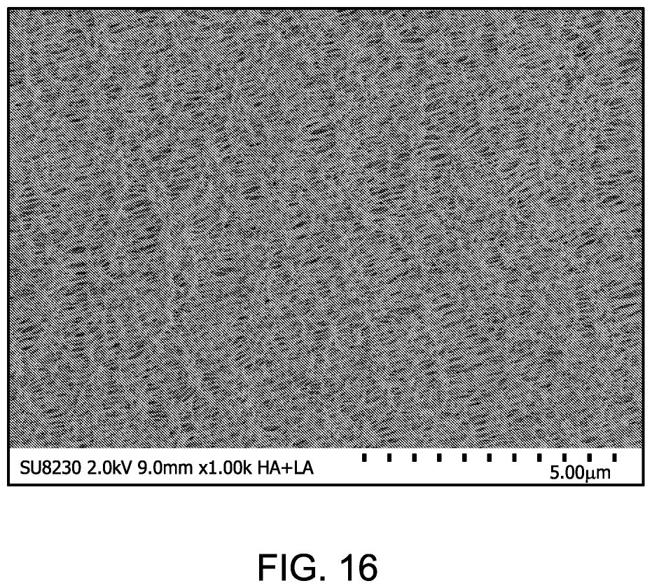

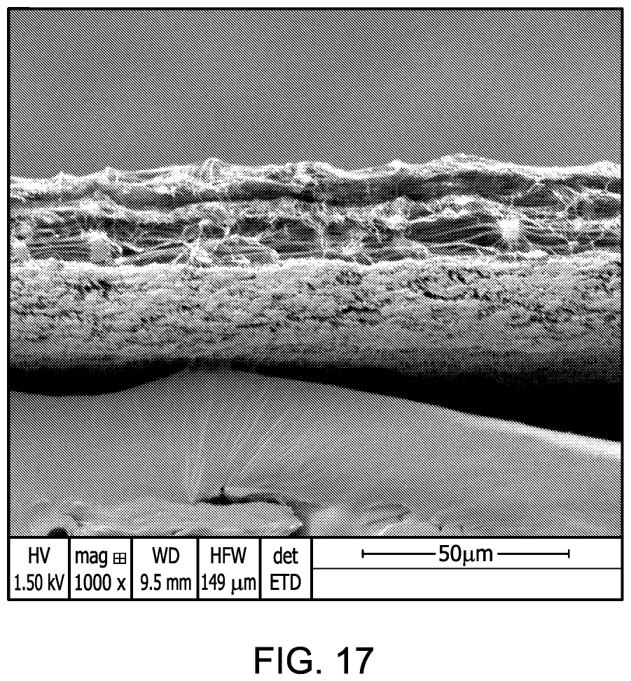

An implantable membrane construct having two distinct layers was constructed. In particular, the two layer composite was prepared by layering, drying, co-expanding, and then heat treating a first expanded polytetrafluoroethylene (ePTFE) layer consisting of a dry expanded tape prepared according to the teachings of U.S. Pat. No. 3,953,566 to Gore (Mesenchymal Mitigation Layer) and a second polytetrafluoroethylene (PTFE) layer consisting of a paste extruded, calendered and distended tape prepared according to the teachings of U.S. Pat. No. 3,953,566 to Gore (Cell Impermeable Layer). The membrane construct had a MPS of 0.65 microns, which provided cell impermeability while maintaining transport of oxygen and nutrients therethrough. The cell impermeable tight layer was oriented to the host facing side of the device and had a surface roughness of 0.64 microns. The open first layer of the membrane construct had a cell permeable surface with a surface roughness of 0.97 microns which was oriented towards the lumen and prevented the formation and spread of mesenchymal cells at the luminal interface. A representative scanning electron micrograph (SEM) of the surfaces of this membrane is shown in FIGS. 15 (lumen-facing) and 16 (host-facing). A representative scanning electron micrograph (SEM) of the cross-section of this membrane construct is shown in FIG. 17.

### Characterization of the Biocompatible Membrane Construct

The membrane construct was evaluated and characterized for the relevant parameters necessary for the intended function. Parameters are marked as “N/A” if they are not relevant for that specific function. Parameters are marked as “-” if they are practically unobtainable as a result of how the layers of the construct were processed. The methods used for the characterization of the relevant parameters were performed in accordance with the methods described in “Test Methods” section set forth. The results are summarized in Table 4.

TABLE 4

| Relevant Properties: | units | Measured Value |
|---|---|---|
| Surface Roughness (Sa), Lumen-Facing Side | μm | 0.97 |
| Surface Roughness (Sa), Host-Facing Side | μm | 0.64 |
| Maximum Pore Size (MPS) | μm | 0.65 |
| Thickness, Mesenchymal Mitigation Layer | μm | 27.3 |
| Mean Pore Size | μm | 0.16 |
| Tensile strength | N/mm | 0.33 |
| Total Thickness | μm | 48.9 |
| Z-strength | kPa | 330 |
| Porosity | % | 88.0 |
| Feature Spacing, Lumen-Facing Side | μm | 9.7 |
| Feature Spacing, Host-Facing Side | μm | N/A |

### Evaluation of the Biocompatible Membrane Construct

The implantable membrane composite was thermally welded into a device form in accordance with the Integration of Biocompatible Membrane into a Device Form set forth in the Test Methods section.

The device was evaluated in vivo in accordance with the In Vivo Nude Rat Study set forth in the Test Methods section.

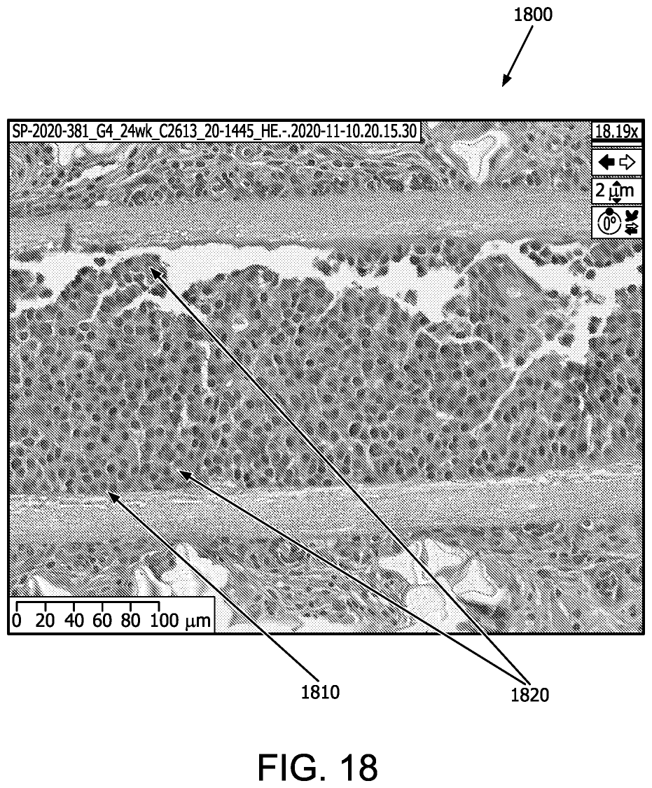

The histopathology image set forth in FIG. 18 shows the implantable membrane construct where the cell impermeable surface (second layer) faced the host tissue, and the cell permeable (first layer) surface faced the graft cell population. The presence of mesenchymal cells was not observed at the graft cell population interface (i.e., luminal interface).

FIG. 18 depicts viable functional cells 1820 adjacent to the luminal interface 1810 of the implantable membrane construct 1800 at the 25-week timepoint.

### Example 3

### Manufacturing of the Implantable Membrane Construct

An implantable membrane construct having three distinct layers was constructed. The three layer composite was prepared by layering, drying, co-expanding, and then heat treating a first PTFE layer consisting of a paste extruded calendered tape prepared according to the teachings of U.S. Pat. No. 3,953,566 to Gore (Mesenchymal Mitigation Layer), a second ePTFE layer consisting of a dry, biaxially expanded membrane prepared according to the teachings of U.S. Pat. No. 3,953,566 to Gore (Cell Impermeable Layer), and a third PTFE layer consisting of a paste extruded calendered tape prepared according to the teachings of U.S. Pat. No. 3,953,566 to Gore (Vascularization Layer).

The implantable membrane construct was a three-layer composite comprised of a tight, cell impermeable second layer and two open, cell permeable layers (i.e., first and third layers)) on opposing sides of the second layer. The membrane construct had a MPS of 0.26 microns, which provided cell impermeability while maintaining transport of oxygen and nutrients therethrough. The third layer of the membrane construct had a cell permeable surface that enabled vascularization oriented to the host facing side of the device with a surface roughness of 3.9 microns. The first layer of the membrane construct had a cell permeable surface oriented on the lumen side of the device that prevented the formation and spread of mesenchymal cells at the luminal interface with a surface roughness of 2.5 microns.

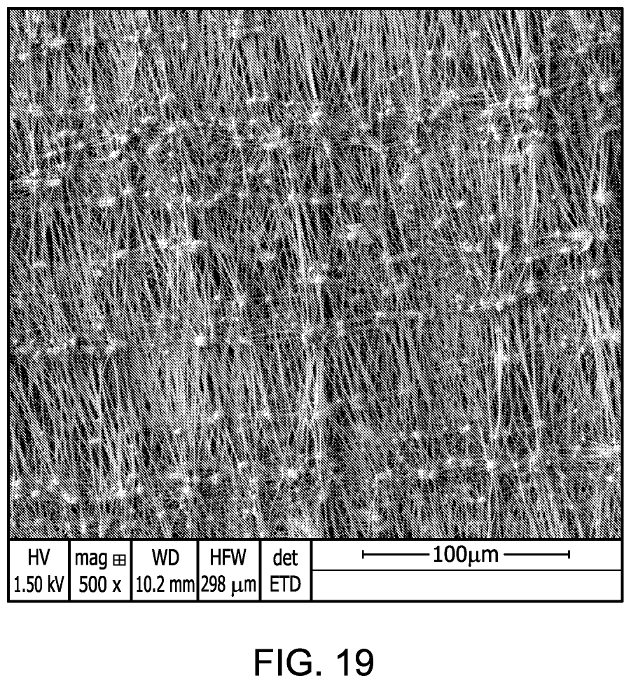

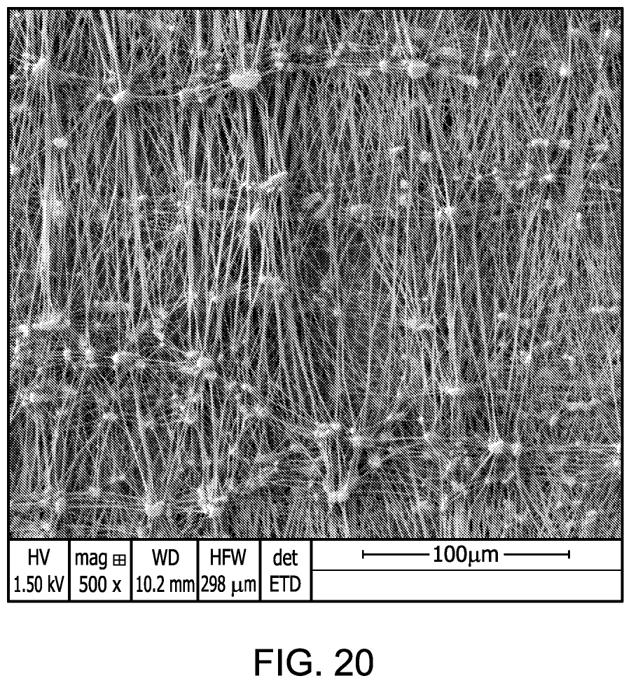

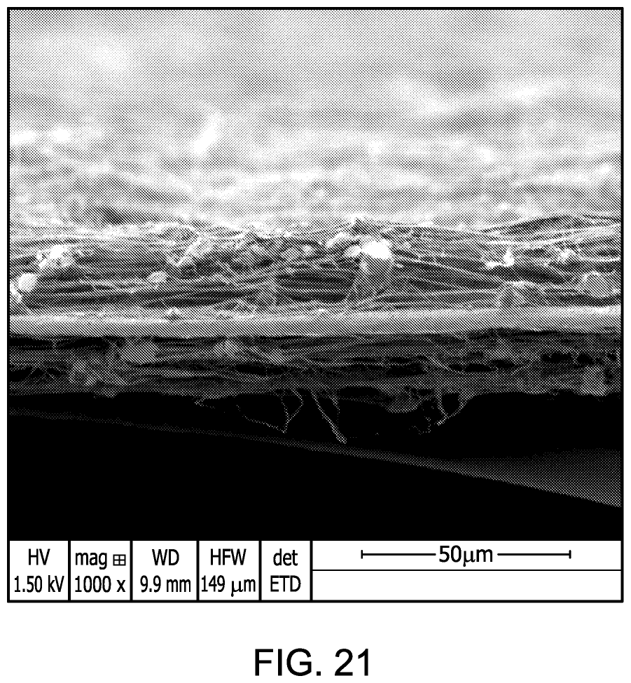

A representative scanning electron micrograph (SEM) of the surfaces of this membrane is shown in FIGS. 19 (lumen-facing) and 20 (host-facing). A representative scanning electron micrograph (SEM) of the cross-section of this membrane construct is shown in FIG. 21.

### Characterization of the Biocompatible Membrane Construct

The membrane construct was evaluated and characterized for the relevant parameters necessary for the intended function. Parameters are marked as “N/A” if they are not relevant for that specific function. Parameters are marked as “-” if they are practically unobtainable as a result of how the layers of the construct were processed. The methods used for the characterization of the relevant parameters were performed in accordance with the methods described in “Test Methods” section. The results are summarized in Table 5.

TABLE 5

| Relevant Properties: | units | Measured Value |
|---|---|---|
| Surface Roughness (Sa), Lumen-Facing Side | μm | 2.5 |
| Surface Roughness (Sa), Host-Facing Side | μm | 3.9 |
| Maximum Pore Size (MPS) | μm | 0.26 |
| Thickness, Mesenchymal Mitigation Layer | μm | 11.9 |
| Mean Pore Size | μm | 0.13 |
| Tensile strength | N/mm | 0.33 |
| Total Thickness | μm | 30.4 |
| Z-strength | kPa | 500 |
| Porosity | % | 85.3 |
| Feature Spacing, Lumen-Facing Side | μm | 16.0 |
| Feature Spacing, Host-Facing Side | μm | 19.8 |

### Evaluation of the Biocompatible Membrane Construct

The implantable membrane construct was thermally welded into a device form in accordance with the Integration of Biocompatible Membrane into a Device Form set forth in the Test Methods section.

The device was evaluated in vivo in accordance with the In Vivo Nude Rat Study set forth in the Test Methods section.

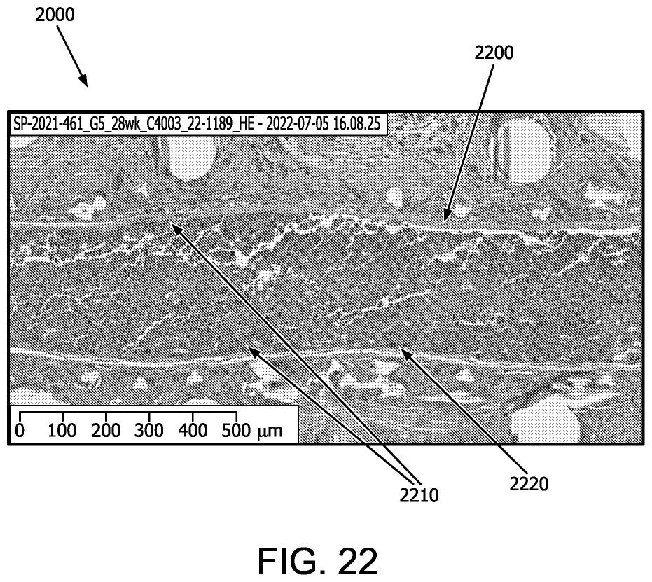

The histopathology image 2000 shown in FIG. 22 depicts an implantable membrane construct 2200, where the first cell permeable (open layer) surface faces the graft cell population. It was observed that mesenchymal cells were not present at the luminal interface 2220. FIG. 22 demonstrates viable functional cells 2210 adjacent to the luminal interface 2220 of the implantable membrane construct 2200 at the 28-week timepoint.

### Example 4

### Manufacturing of the Implantable Membrane Construct

An implantable membrane construct having three distinct layers was constructed. The three layer composite was prepared by layering, drying, co-expanding, and then heat treating a first ePTFE layer consisting of a dry expanded tape prepared according to the teachings of U.S. Pat. No. 3,953,566 to Gore (Mesenchymal Mitigation Layer), a second PTFE layer consisting of a paste extruded, calendered and distended tape prepared according to the teachings of U.S. Pat. No. 3,953,566 to Gore (Cell Impermeable Layer), and a third PTFE layer consisting of a dry expanded tape prepared according to the teachings of U.S. Pat. No. 3,953,566 to Gore (Vascularization Layer). The implantable membrane construct was a three-layer composite comprised of a tight, cell impermeable second layer and two open, cell permeable layers (i.e., first and third layers) on opposing sides of the second layer. The membrane construct had a MPS of 0.79 microns, which provided cell impermeability while maintaining transport of oxygen and nutrients therethrough. The third layer of the membrane construct had a cell permeable surface with a surface roughness of 1.12, which prevented the formation and spread of mesenchymal cells at the luminal interface.

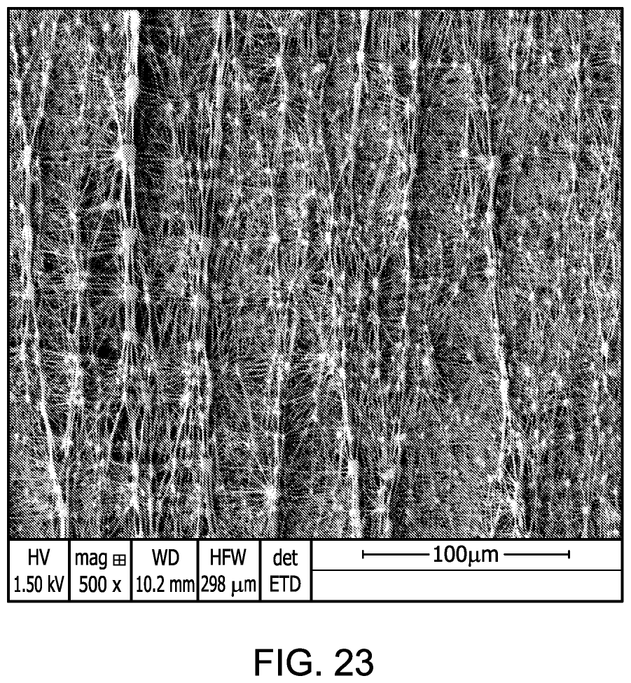

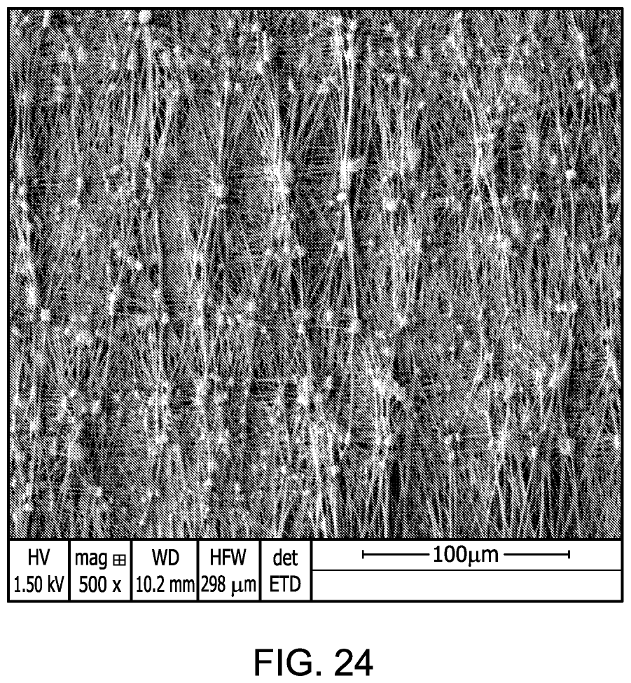

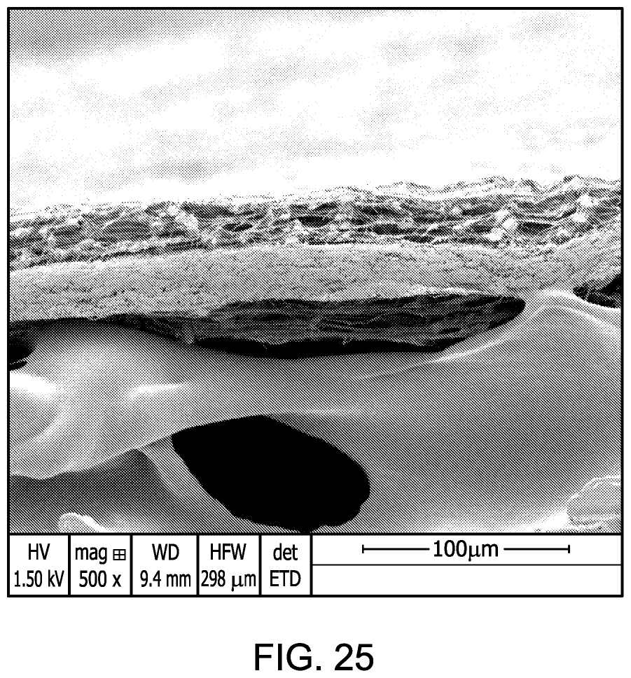

A representative scanning electron micrograph (SEM) of the surfaces of this membrane is shown in FIGS. 23 (lumen-facing) and 24 (host-facing). A representative scanning electron micrograph (SEM) of the cross-section of this membrane construct is shown in FIG. 25.

### Characterization of the Biocompatible Membrane Construct

The membrane construct was evaluated and characterized for the relevant parameters necessary for the intended function. Parameters are marked as “N/A” if they are not relevant for that specific function. Parameters are marked as “-” if they are practically unobtainable as a result of how the layers of the construct were processed. The methods used for the characterization of the relevant parameters were performed in accordance with the methods described in “Test Methods” section. The results are summarized in Table 6.

TABLE 6

| Relevant Properties: | units | Measured Value |
|---|---|---|
| Surface Roughness (Sa), Lumen-Facing Side | μm | 1.12 |
| Surface Roughness (Sa), Host-Facing Side | μm | 1.95 |
| Maximum Pore Size (MPS) | μm | 0.79 |
| Thickness, Mesenchymal Mitigation Layer | μm | 28.8 |
| Mean Pore Size | μm | 0.27 |
| Tensile strength | N/mm | 0.75 |
| Total Thickness | μm | 64.4 |
| Z-strength | kPa | 235.4 |
| Porosity | % | 92.3 |
| Feature Spacing, Lumen-Facing Side | μm | 9.4 |
| Feature Spacing, Host-Facing Side | μm | 12.9 |

### Evaluation of the Biocompatible Membrane Construct

The implantable membrane construct was thermally welded into a device form in accordance with the Integration of Biocompatible Membrane into a Device Form set forth in the Test Methods section.

The device was evaluated in vivo in accordance with the In Vivo Nude Rat Study set forth in the Test Methods section.

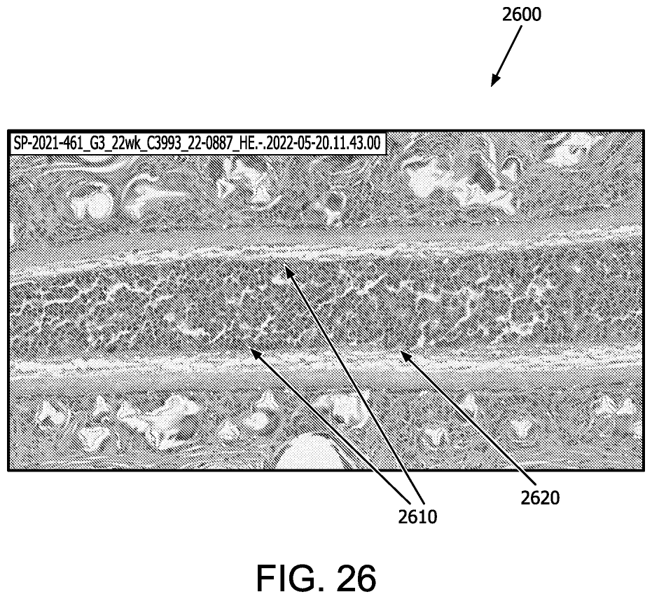

The histopathology image shown in FIG. 26 is an implantable membrane construct 2600, where the first cell permeable (open layer) surface faces the graft cell population. It was observed that mesenchymal cells were not present at the luminal interface 2620. FIG. 26 demonstrates viable functional cells 2610 adjacent to the luminal interface 2620 of the implantable membrane construct 2600 at the 28-week timepoint.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. An implantable membrane construct comprising:
a first layer having a maximum pore size (MPS) less than about 2 microns and opposing sides, wherein each said opposing side has a surface roughness of at least 0.5 microns.

2. The implantable membrane construct of claim 1, wherein the first layer comprises a polymer membrane.

3. (canceled)

4. (canceled)

5. The implantable membrane construct of claim 1, comprising a surface coating at least partially on the first layer, wherein the surface coating includes one or more members selected from antimicrobial agents, antibodies, pharmaceuticals, biologically active molecules, and a hydrophilic coating.

6. The implantable membrane construct of claim 1, comprising a frame positioned around the perimeter of the first layer.

7. The implantable membrane construct of claim 1, comprising multiple regions, each said region having a microstructure.

8. An implantable membrane construct comprising:
a first layer; and
second layer having a maximum pore size (MPS) less than about 2.0 microns, wherein the second layer is positioned on the first layer,
wherein the first layer has a first externally facing side and the second layer has a second externally facing side, wherein the first externally facing side and the second externally facing side each has a surface roughness (Sa) of at least 0.5 microns.

9. The implantable membrane construct of claim 8, wherein the first layer is cell permeable, and the second layer is cell impermeable.

10. The implantable membrane construct of claim 8, wherein the membrane construct has a total thickness from about 15 microns to about 150 microns.

11. The implantable membrane construct of claim 8, comprising a mean flow pore size greater than about 0.1 microns.

12. The implantable membrane construct of claim 8, wherein the tensile strength in the weakest direction is greater than about 0.15 N/mm.

13. The implantable membrane construct of claim 8, comprising a reinforcing component.

14. (canceled)

15. (canceled)

16. The implantable membrane construct of claim 8, wherein at least one of the first layer and the second layer includes a polymer, a fluoropolymer membrane, a non-fluoropolymer membrane, a woven textile, a non-woven textile, woven or non-woven collections of fibers or yarns, fibrous matrices, and combinations thereof.

17. (canceled)

18. The implantable membrane construct of claim 8, wherein the first layer and the second layer are intimately bonded.

19. The implantable membrane construct of claim 8, wherein the coefficient of variation of the surface roughness (Sa) of opposing sides in the implantable membrane construct is less than 15%.

20. An implantable membrane construct comprising:
a first layer having a first externally facing side with a surface roughness (Sa) of at least 0.5 microns,
a second layer positioned on the first layer on a side opposing the first externally facing side; and
a third layer positioned on the second layer such that the second layer is positioned between the first layer and the third layer, wherein the third layer has a second externally facing side having a surface roughness (Sa) of at least 0.5 microns,
wherein the implantable membrane has a maximum pore size less than about 2.0 microns.

21. The implantable membrane construct of claim 20, wherein the first layer is cell permeable and the second and third layers are cell impermeable.

22. The implantable membrane construct of claim 20, comprising a mean flow pore size greater than about 0.1 microns.

23. The implantable membrane construct of claim 20, wherein the first layer has a thickness from about 2 microns to about 100 microns.

24. The implantable membrane construct of claim 20, wherein the surface roughness (Sa) of the first layer and the third layer has a coefficient of variation that is less than 15%.

25. The implantable membrane construct of claim 20, wherein the feature spacing in the third layer is greater than about 3 microns.

26-36. (canceled)

37. A cell encapsulation device comprising:
an implantable membrane construct having a luminal interface with a surface roughness of at least 0.5 microns, an exterior surface with the surface roughness of at least 0.5 microns, and a maximum pore size (MPS) of less than about 2 microns.

38-72. (canceled)
